(19) United States
(12) Patent Application Publication
(10) Pub. No.: US 2008/0081824 A1
(43) Pub. Date: Apr. 3, 2008

(54) Substituted piperidines as modulators of chemokine receptor activity

(75) Inventors: Changsheng Zheng, Wilmington, DE (US); Dean A. Wacker, Yardley, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/541,311

(22) Filed: Sep. 29, 2006

(51) Int. Cl. C07D 401/02; C07D 211/56; A61K 31/445; A61K 31/454

(52) U.S. Cl. 514/317; 546/207; 546/223; 514/326; 546/209; 546/210

(57) ABSTRACT

The present application describes modulators of CCR3 of formula (I):

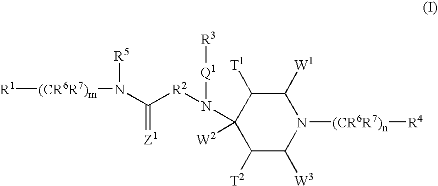

or stereoisomers or prodrugs or pharmaceutically acceptable salts thereof, wherein m, n, Q¹, R¹, R², R³, R⁴, R⁵, R⁶, R⁷, T¹, T², W¹, W², W³ and Z¹ are as defined herein. In addition, methods of treating and preventing inflammatory diseases such as asthma and allergic diseases, as well as autoimmune pathologies such as rheumatoid arthritis and atherosclerosis using said modulators are disclosed.

### FIELD OF THE INVENTION

This invention relates generally to modulators of chemokine receptor activity, pharmaceutical compositions containing the same, and methods of using the same as agents for treatment and prevention of inflammatory diseases such as asthma and allergic diseases, as well as autoimmune pathologies such as rheumatoid arthritis and atherosclerosis.

### BACKGROUND OF THE INVENTION

Chemokines are chemotactic cytokines, of molecular weight 6-15 kDa, that are released by a wide variety of cells to attract and activate, among other cell types, macrophages, T and B lymphocytes, eosinophils, basophils and neutrophils (reviewed in Luster, New Eng. J. Med., 338, 436-445 (1998) and Rollins, Blood, 90, 909-928 (1997)). There are two major classes of chemokines, CXC and CC, depending on whether the first two cysteines in the amino acid sequence are separated by a single amino acid (CXC) or are adjacent (CC). The CXC chemokines, such as interleukin-8 (IL-8), neutrophil-activating protein-2 (NAP-2) and melanoma growth stimulatory activity protein (MGSA) are chemotactic primarily for neutrophils and T lymphocytes, whereas the CC chemokines, such as RANTES, MIP-1α, MIP-1β, the monocyte chemotactic proteins (MCP-1, MCP-2, MCP-3, MCP-4, and MCP-5) and the eotaxins (-1, -2, and -3) are chemotactic for, among other cell types, macrophages, T lymphocytes, eosinophils, dendritic cells, and basophils. There also exist the chemokines lymphotactin-1, lymphotactin-2 (both C chemokines), and fractalkine (a CXXXC chemokine) that do not fall into either of the major chemokine subfamilies.

The chemokines bind to specific cell-surface receptors belonging to the family of G-protein-coupled seven-transmembrane-domain proteins (reviewed in Horuk, Trends Pharm. Sci., 15, 159-165 (1994)) which are termed “chemokine receptors.” On binding their cognate ligands, chemokine receptors transduce an intracellular signal through the associated trimeric G proteins, resulting in, among other responses, a rapid increase in intracellular calcium concentration, changes in cell shape, increased expression of cellular adhesion molecules, degranulation, and promotion of cell migration. There are at least ten human chemokine receptors that bind or respond to CC chemokines with the following characteristic patterns: CCR-1 (or “CKR-1” or “CC-CKR-1”) [MIP-1α, MCP-3, MCP-4, RANTES] (Ben-Barruch, et al., Cell, 72, 415-425 (1993), Luster, New Eng. J. Med., 338, 436-445 (1998)); CCR-2A and CCR-2B (or “CKR-2A”/“CKR-2B” or “CC-CKR-2A”/“CC-CKR-2B”) [MCP-1, MCP-2, MCP-3, MCP-4, MCP-5] (Charo et al., Proc. Natl. Acad. Sci. USA, 91, 2752-2756 (1994), Luster, New Eng. J. Med., 338, 436-445 (1998)); CCR-3 (or “CKR-3” or “CC-CKR-3”) [eotaxin-1, eotaxin-2, RANTES, MCP-3, MCP-4] (Combadiere, et al., J. Biol. Chem., 270, 16491-16494 (1995), Luster, New Eng. J. Med., 338, 436-445 (1998)); CCR-4 (or “CKR-4” or “CC-CKR-4”) [TARC, MIP-1α, RANTES, MCP-1] (Power et al., J. Biol. Chem., 270, 19495-19500 (1995), Luster, New Eng. J. Med., 338, 436-445 (1998)); CCR-5 (or “CKR-5” OR “CC-CKR-5”) [MIP-1α, RANTES, MIP-1β] (Sanson, et al., Biochemistry, 35, 3362-3367 (1996)); CCR-6 (or “CKR-6” or “CC-CKR-6”) [LARC] (Baba et al., J. Biol. Chem., 272, 14893-14898 (1997)); CCR-7 (or “CKR-7” or “CC-CKR-7”) [ELC] (Yoshie et al., J. Leukoc. Biol. 62, 634-644 (1997)); CCR-8 (or “CKR-8” or “CC-CKR-8”) [I-309, TARC, MIP-1β] (Napolitano et al., J. Immunol., 157, 2759-2763 (1996), Bernardini et al., Eur. J. Immunol., 28, 582-588 (1998)); and CCR-10 (or “CKR-10” or “CC-CKR-10”) [MCP-1, MCP-3] (Bonini et al, DNA and Cell Biol., 16, 1249-1256 (1997)).

In addition to the mammalian chemokine receptors, mammalian cytomegaloviruses, herpesviruses and poxviruses have been shown to express, in infected cells, proteins with the binding properties of chemokine receptors (reviewed by Wells and Schwartz, Curr. Opin. Biotech., 8, 741-748 (1997)). Human CC chemokines, such as RANTES and MCP-3, can cause rapid mobilization of calcium via these virally encoded receptors. Receptor expression may be permissive for infection by allowing for the subversion of normal immune system surveillance and response to infection. Additionally, human chemokine receptors, such as CXCR4, CCR2, CCR3, CCR5 and CCR8, can act as co-receptors for the infection of mammalian cells by microbes as with, for example, the human immunodeficiency viruses (HIV).

Chemokine receptors have been implicated as being important mediators of inflammatory, infectious, and immunoregulatory disorders and diseases, including asthma and allergic diseases, as well as autoimmune pathologies such as rheumatoid arthritis and atherosclerosis. For example, the chemokine receptor CCR-3 plays a pivotal role in attracting eosinophils to sites of allergic inflammation and in subsequently activating these cells. The chemokine ligands for CCR-3 induce a rapid increase in intracellular calcium concentration, increased expression of cellular adhesion molecules, cellular degranulation, and the promotion of eosinophil migration. Accordingly, agents which modulate chemokine receptors would be useful in such disorders and diseases. In addition, agents which modulate chemokine receptors would also be useful in infectious diseases such as by blocking infection of CCR3 expressing cells by HIV or in preventing the manipulation of immune cellular responses by viruses such as cytomegaloviruses.

### SUMMARY OF THE INVENTION

Accordingly, the present invention provides novel agonists or antagonists of CCR-3, or pharmaceutically acceptable salts or prodrugs thereof.

The present invention provides pharmaceutical compositions comprising a pharmaceutically acceptable carrier and a therapeutically effective amount of at least one of the compounds of the present invention or a pharmaceutically acceptable salt or prodrug form thereof.

The present invention provides a method for treating inflammatory diseases and allergic disorders comprising administering to a host in need of such treatment a therapeutically effective amount of at least one of the compounds of the present invention or a pharmaceutically acceptable salt or prodrug form thereof.

The present invention provides novel substituted piperidines, for example, 4-amino substituted piperidines, for use in therapy.

Further, the present invention provides the use of novel substituted piperidines, for example, 4-amino-substituted piperidines, for the manufacture of a medicament for the treatment of allergic disorders.

These and other aspects, which will become apparent during the following detailed description, have been achieved by the inventors' discovery that compound of formula (I):

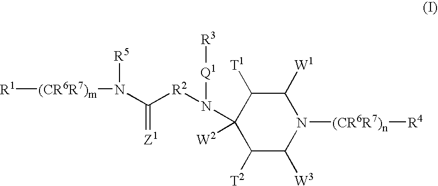

or stereoisomers or prodrugs or pharmaceutically acceptable salts thereof, wherein m, n, Q¹, R¹, R², R³, R⁴, R⁵, R⁶, R⁷, T¹, T², W¹, W², W³ and Z¹ are defined below, and are effective modulators of chemokine activity.

### DETAILED DESCRIPTION OF PREFERRED EMBODIMENTS

In one embodiment, the present invention discloses novel compounds of formula (I):

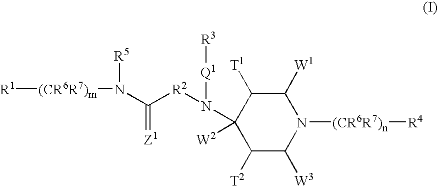

or a stereoisomer or prodrug or pharmaceutically acceptable salt from thereof, wherein:

Q¹ is independently selected from C═O, C(O)₂, CON(R⁸)R⁸, S(O), S(O)₂ and S(O)₂N(R⁸)R⁸;

Z¹ is independently selected from O, S, N(R⁸), C(CN)₂, CH(NO₂), and CH(CN);

R¹ is independently selected from hydrogen, C₁-C₆ alkyl, (CH₂)_{q}C₃-C₆ cycloalkyl, C₃-C₆ cycloalkyl(CH₂)ᵣ, aryl and heterocyclyl, wherein the C₁-C₆ alkyl, (CH₂)_{q}C₃-C₆ cycloalkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁸)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;

R² is independently selected from C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ, wherein the C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;

R³ is independently selected from hydrogen, C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ wherein the C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;

R⁴ is independently selected from C₁-C₆ alkyl, (CH₂)_{q}C₃-C₆ cycloalkyl, C₃-C₆ cycloalkyl(CH₂)ᵣ, aryl and heterocyclyl, wherein the C₁-C₆ alkyl, (CH₂)_{q}C₃-C₆ cycloalkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;

R⁵ is selected from hydrogen, C₁₋₈ alkyl, C₃₋₈ alkenyl, C₃₋₈ alkynyl, (CH₂)ᵣC₃₋₆ cycloalkyl, (CH₂)_{q}C(O)R¹⁶, (CH₂)_{q}C(O)NR¹⁵R¹⁵, (CH₂)_{q}C(O)OR¹⁶, (CH₂)ᵣaryl optionally substituted with 0-3 R¹⁷, and (CH₂)ᵣheterocyclyl optionally substituted with 0-3 R¹⁷;

R⁶ and R⁷, at each occurrence, are independently selected from hydrogen, C₁₋₆ alkyl, (CH₂)ᵣC₃₋₆ cycloalkyl, and (CH₂)ᵣphenyl, or optionally, R⁶ and R⁷ may be taken together with the carbon to which both are attached to form a C₃₋₆ cycloalkyl ring;

R⁸, at each occurrence, is independently selected from hydrogen, C₁-C₆ alkyl, C₂₋₈ alkenyl, C₂₋₈ alkynyl, (CH₂)ᵣC₃-C₆ cycloalkyl, (CH₂)ᵣC(O)R¹⁹, (CH₂)ᵣC(O)N(R^{18a})R^{18b}, (CH₂)ᵣC(O)OR¹⁹, (CH₂)ᵣS(O)₂R¹⁹, (CH₂)ᵣS(O)₂N(R^{18a})R^{18b}, OR¹⁹, CN, NO₂, and (CH₂)ᵣphenyl substituted with 0-3 R¹⁸;

R⁹, at each occurrence, is independently selected from hydrogen, C₁₋₆ alkyl, C₂₋₈ alkenyl, C₂₋₈ alkynyl, (CH₂)ᵣaryl substituted with 0-5 R¹³, and a (CH₂)ᵣheterocyclyl containing 1-4 heteroatoms selected from N, O, and S, substituted with 0-2 R¹³;

R¹⁰, at each occurrence, is independently selected from C₁₋₆ alkyl, C₂₋₈ alkenyl, C₂₋₈ alkynyl, (CH₂)ᵣaryl substituted with 0-3 R¹³, and a (CH₂)ᵣheterocyclyl containing 1-4 heteroatoms selected from N, O, and S, substituted with 0-2 R¹³;

R¹², at each occurrence, is independently selected from C₂₋₈ alkenyl, C₂₋₈ alkynyl, C₁₋₆ alkyl substituted with 0-3 R¹³, (CH₂)ᵣaryl substituted with 0-3 R¹³, and a (CH₂)ᵣheterocyclyl containing 1-4 heteroatoms selected from N, O, and S, substituted with 0-3 R¹³;

R¹³, at each occurrence, is independently selected from C₁₋₆ alkyl, C₂₋₈ alkenyl, C₂₋₈ alkynyl, (CH₂)ᵣC₃₋₆ cycloalkyl, Cl, F, Br, I, CN, NO₂, (CF₂)ᵣCF₃, (CH₂)ᵣOC₁₋₅ alkyl, OH, SH, (CH₂)ᵣSC₁₋₅ alkyl, and (CH₂)ᵣNR¹⁴R¹⁴;

R¹⁴, at each occurrence, is independently selected from H, C₁₋₅ alkyl, and C₃₋₆ cycloalkyl, and phenyl;

R¹⁵, at each occurrence, is independently selected from H, C₁₋₆ alkyl, (CH₂)ᵣC₃₋₆ cycloalkyl, and (CH₂)ᵣphenyl;

R¹⁶, at each occurrence, is independently selected from C₁₋₆ alkyl, C₂₋₈ alkenyl, (CH₂)ᵣC₃₋₆ cycloalkyl, C₂₋₈ alkynyl, and (CH₂)ᵣphenyl;

R¹⁷, at each occurrence, is independently selected from C₁₋₆ alkyl, C₂₋₈ alkenyl, C₂₋₈ alkynyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣNR¹⁵R¹⁵, and (CH₂)ᵣphenyl;

R¹⁸, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣS(O)C₁₋₅ alkyl, (CH₂)ᵣS(O)₂C₁₋₅ alkyl, (CH₂)ᵣS(O)₂N(R^{18a})R^{18b}, (CH₂)ᵣN(R^{18c})C(O)C₁₋₅ alkyl (CH₂)ᵣN(R^{18c})S(O)₂C₁₋₅ alkyl, (CH₂)ᵣC(O)N(R^{18a})R^{18b}, (CH₂)ᵣC(O)OC₁₋₅ alkyl, (CH₂)ᵣC(O)C₁₋₅ alkyl, and (CH₂)ᵣN(R^{18a})R^{18b};

R^{18a}, R^{18b}, and R^{18c}, at each occurrence, are independently selected from H, C₁₋₆ alkyl, (CH₂)ᵣC₃₋₆ cycloalkyl, and (CH₂)ᵣphenyl;

R¹⁹, at each occurrence, is independently selected from hydrogen, C₁₋₆ alkyl, (CH₂)ᵣC₃₋₆ cycloalkyl, and (CH₂)ᵣphenyl;

T¹ and T² are independently selected from hydrogen, C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;

W¹, W² and W³ are independently selected from hydrogen, C₁₋₈ alkyl, (CH₂)ᵣC₃₋₆ cycloalkyl, (CHR⁸)_{q}NR⁹R⁹, (CHR⁸)_{q}OH, (CHR⁸)_{q}O(CHR⁸)ᵣR¹², (CHR⁸)_{q}SH, (CHR⁸)ᵣC(O)H, (CHR⁸)_{q}S(CHR⁸)ᵣR¹², (CHR⁸)ᵣC(O)OH, (CHR⁸)ᵣC(O)(CHR⁶)ᵣR¹, (CHR⁸)ᵣC(O)NR⁹R⁹, (CHR⁸)_{q}NR¹⁴C(O)(CHR⁸)ᵣR¹⁰, (CHR⁸)ᵣC(O)O(CHR⁸)ᵣR¹², (CHR⁸)_{q}OC(O)(CHR⁸)ᵣR¹⁰, (CHR⁸)ᵣC(═NR¹⁴)NR⁹R⁹, (CHR⁸)_{q}NHC(═NR¹⁴)NR¹⁴R¹⁴, (CHR⁸)_{q}S(O)(CHR⁸)ᵣR¹⁰, (CHR⁸)_{q}S(O)₂(CHR⁸)ᵣR¹⁰, (CHR⁸)_{q}S(O)₂NR⁹R⁹, (CHR⁸)_{q}NR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;

m is 0-3;

n is 0-3;

q is 1-5; and

r is 0-5.

In another embodiment, compounds of formula (I) are those compounds in which:

Q¹ is independently selected from C═O, C(O)₂, and CON(R⁸)R⁸;

Z¹ is independently selected from O and S;

R¹ is independently selected from aryl and heterocyclyl, wherein the aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R² is independently selected from C₁-C₆ alkyl, C₃-C₆ cycloalkyl(CH₂)ᵣ and aryl(CH₂)ᵣ, wherein the C₁-C₆ alkyl, C₃-C₆ cycloalkyl(CH₂)ᵣ and aryl(CH₂)ᵣ may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R³ is independently selected from hydrogen, C₁-C₆ alkyl, C₃-C₆ cycloalkyl(CH₂)ᵣ, aryl and heterocyclyl, wherein the C₁-C₆ alkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R⁴ is independently selected from C₃-C₆ cycloalkyl(CH₂)ᵣ, aryl and heterocyclyl, wherein the C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R⁵ is selected from hydrogen and C₁₋₈ alkyl;

R⁶ and R⁷, at each occurrence, are independently selected from hydrogen and C₁₋₆ alkyl;

R⁸, at each occurrence, is independently selected from hydrogen, C₁-C₆ alkyl, (CH₂)ᵣC₃-C₆ cycloalkyl, (CH₂)ᵣC(O)R¹⁹, (CH₂)ᵣC(O)N(R^{18a})R^{18b}, (CH₂)ᵣC(O)OR¹⁹, (CH₂)ᵣS(O)₂R¹⁹, (CH₂)ᵣS(O)₂N(R^{18a})R^{18b}, OR¹⁹, CN, NO₂, and (CH₂)ᵣphenyl substituted with 0-3 R¹⁸;

R⁹, at each occurrence, is independently selected from hydrogen, C₁₋₆ alkyl, (CH₂)ᵣaryl substituted with 0-5 R¹³, and a (CH₂)ᵣheterocyclyl containing 1-4 heteroatoms selected from N, O, and S, substituted with 0-2 R¹³;

R¹⁰, at each occurrence, is independently selected from C₁₋₆ alkyl, (CH₂)ᵣaryl substituted with 0-3 R¹³, and a (CH₂)ᵣheterocyclyl containing 1-4 heteroatoms selected from N, O, and S, substituted with 0-2 R¹³;

R¹², at each occurrence, is independently selected from C₁₋₆ alkyl substituted with 0-3 R¹³, (CH₂)ᵣaryl substituted with 0-3 R¹³, and a (CH₂)ᵣheterocyclyl containing 1-4 heteroatoms selected from N, O, and S, substituted with 0-3 R¹³;

R¹³, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, OH, SH, (CH₂)ᵣSC₁₋₅ alkyl, and (CH₂)ᵣNR¹⁴R¹⁴;

R¹⁴, at each occurrence, is independently selected from H, C₁₋₅ alkyl, C₃₋₆ cycloalkyl and phenyl;

R¹⁵, at each occurrence, is independently selected from H, C₁₋₆ alkyl, (CH₂)ᵣC₃₋₆ cycloalkyl, and (CH₂)ᵣphenyl;

R¹⁶, at each occurrence, is independently selected from C₁₋₆ alkyl, (CH₂)ᵣC₃₋₆ cycloalkyl and (CH₂)ᵣphenyl;

R¹⁷, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣNR¹⁵R¹⁵ and (CH₂)ᵣphenyl;

R¹⁸, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣS(O)C₁₋₅ alkyl, (CH₂)ᵣS(O)₂C₁₋₅ alkyl, (CH₂)ᵣS(O)₂N(R^{18a})R^{18b}, (CH₂)ᵣN(R^{18c})C(O)C₁₋₅ alkyl (CH₂)ᵣN(R^{18c})S(O)₂C₁₋₅ alkyl, (CH₂)ᵣC(O)N(R^{18a})R^{18b}, (CH₂)ᵣC(O)OC₁₋₅ alkyl, (CH₂)ᵣC(O)C₁₋₅ alkyl, and (CH₂)ᵣN(R^{18a})R^{18b};

R^{18a}, R^{18b}, and R^{18c}, at each occurrence, are independently selected from H, C₁₋₆ alkyl, (CH₂)ᵣC₃₋₆ cycloalkyl and (CH₂)ᵣphenyl;

R¹⁹, at each occurrence, is independently selected from hydrogen, C₁₋₆ alkyl (CH₂)ᵣC₃₋₆ cycloalkyl, and (CH₂)ᵣphenyl;

T¹ and T² are independently selected from hydrogen, C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, and (CH₂)ᵣphenyl substituted with 0-3 R¹³;

W¹, W² and W³ are independently selected from hydrogen, C₁₋₈ alkyl, (CH₂)ᵣC₃₋₆ cycloalkyl, (CHR⁸)_{q}NR⁹R⁹, (CHR⁸)_{q}OH, (CHR⁸)_{q}O(CHR⁸)ᵣR¹², (CHR⁸)_{q}SH, (CHR⁸)ᵣC(O)H, (CHR⁸)_{q}S(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)_{q}NR¹⁴C(O)(CHR⁸)ᵣR¹⁰, (CHR⁸)ᵣC(O)O(CHR⁸)ᵣR¹², (CHR⁸)_{q}OC(O)(CHR⁸)ᵣR¹⁰, (CHR⁸)ᵣC(═NR¹⁴)NR⁹R⁹, (CHR⁸)_{q}NHC(═NR¹⁴)NR¹⁴R¹⁴, (CHR⁸)_{q}S(O)(CHR⁸)ᵣR¹⁰, (CHR⁸)_{q}S(O)₂(CHR⁸)ᵣR¹⁰, (CHR⁸)_{q}S(O)₂NR⁹R⁹, (CHR⁸)_{q}NR¹⁴S(O)₂(CHR⁸)ᵣR¹⁰, C₁₋₆ haloalkyl, and (CH₂)ᵣphenyl substituted with 0-3 R¹³;

m is 0-2;

n is 0-2;

q is 1-2; and

r is 0-2.

In yet another embodiment, compounds of formula (I) are those in which:

Q¹ is C═O;

Z¹ is O;

R¹ is aryl, wherein the aryl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R² is independently selected from C₁-C₆ alkyl and aryl, wherein the C₁-C₆ alkyl and aryl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R³ is independently selected from C₁-C₆ alkyl, C₃-C₆ cycloalkyl(CH₂)ᵣ, aryl and heterocyclyl, wherein the C₁-C₆ alkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R⁴ is independently selected from aryl and heterocyclyl, wherein the aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;

R⁵ is hydrogen;

R⁶ and R⁷, at each occurrence, are hydrogen;

R⁸, at each occurrence, is independently selected from hydrogen, C₁-C₆ alkyl, (CH₂)ᵣC₃-C₆ cycloalkyl, (CH₂)ᵣC(O)R¹⁹, (CH₂)ᵣC(O)N(R^{18a})R^{18b}, (CH₂)ᵣC(O)OR¹⁹, (CH₂)ᵣS(O)₂R¹⁹, (CH₂)ᵣS(O)₂N(R^{18a})R^{18b}, OR¹⁹, CN, NO₂, and (CH₂)ᵣphenyl substituted with 0-3 R¹⁸;

R⁹, at each occurrence, is independently selected from hydrogen, C₁₋₆ alkyl, (CH₂)ᵣaryl substituted with 0-5 R¹³, and a (CH₂)ᵣheterocyclyl containing 1-4 heteroatoms selected from N, O, and S, substituted with 0-2 R¹³;

R¹⁰, at each occurrence, is independently selected from C₁₋₆ alkyl, (CH₂)ᵣaryl substituted with 0-3 R¹³, and a (CH₂)ᵣheterocyclyl containing 1-4 heteroatoms selected from N, O, and S, substituted with 0-2 R¹³;

R¹², at each occurrence, is independently selected from C₁₋₆ alkyl substituted with 0-3 R¹³, (CH₂)ᵣaryl substituted with 0-3 R¹³, and a (CH₂)ᵣheterocyclyl containing 1-4 heteroatoms selected from N, O, and S, substituted with 0-3 R¹³;

R¹³, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, OH, SH, (CH₂)ᵣSC₁₋₅ alkyl, and (CH₂)ᵣNR¹⁴R¹⁴;

R¹⁴, at each occurrence, is independently selected from H, C₁₋₅ alkyl, C₃₋₆ cycloalkyl and phenyl;

R¹⁵, at each occurrence, is independently selected from H, C₁₋₆ alkyl, (CH₂)ᵣC₃₋₆ cycloalkyl, and (CH₂)ᵣphenyl;

R¹⁶, at each occurrence, is independently selected from C₁₋₆ alkyl, (CH₂)ᵣC₃₋₆ cycloalkyl and (CH₂)ᵣphenyl;

R¹⁷, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣNR¹⁵R¹⁵ and (CH₂)ᵣphenyl;

R¹⁸, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣS(O)C₁₋₅ alkyl, (CH₂)ᵣS(O)₂C₁₋₅ alkyl, (CH₂)ᵣS(O)₂N(R^{18a})R^{18b}, (CH₂)ᵣN(R^{18c})C(O)C₁₋₅ alkyl (CH₂)ᵣN(R^{18c})S(O)₂C₁₋₅ alkyl, (CH₂)ᵣC(O)N(R^{18a})R^{18b}, (CH₂)ᵣC(O)OC₁₋₅ alkyl, (CH₂)ᵣC(O)C₁₋₅ alkyl, and (CH₂)ᵣN(R^{18a})R^{18b};

R^{18a}, R^{18b}, and R^{18c}, at each occurrence, are independently selected from H, C₁₋₆ alkyl, (CH₂)ᵣC₃₋₆ cycloalkyl and (CH₂)ᵣphenyl;

R¹⁹, at each occurrence, is independently selected from hydrogen, C₁₋₆ alkyl (CH₂)ᵣC₃₋₆ cycloalkyl, and (CH₂)ᵣphenyl;

T¹ and T² are independently selected from hydrogen, C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, and (CH₂)ᵣphenyl substituted with 0-3 R¹³;

W¹, W² and W³ are independently selected from hydrogen, C₁₋₈ alkyl, (CH₂)ᵣC₃₋₆ cycloalkyl, (CHR⁸)_{q}NR⁹R⁹, (CHR⁸)_{q}OH, (CHR⁸)_{q}O(CHR⁸)ᵣR¹², (CHR⁸)_{q}SH, (CHR⁸)ᵣC(O)H, (CHR⁸)_{q}S(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)_{q}NR¹⁴C(O)(CHR⁸)ᵣR¹⁰, (CHR⁸)ᵣC(O)O(CHR⁸)ᵣR¹², (CHR⁸)_{q}OC(O)(CHR⁸)ᵣR¹⁰, (CHR⁸)ᵣC(═NR¹⁴)NR⁹R⁹, (CHR⁸)_{q}NHC(═NR¹⁴)NR¹⁴R¹⁴, (CHR⁸)_{q}S(O)(CHR⁸)ᵣR¹⁰, (CHR⁸)_{q}S(O)₂(CHR⁸)ᵣR¹⁰, (CHR⁸)_{q}S(O)₂NR⁹R⁹, (CHR⁸)_{q}NR¹⁴S(O)₂(CHR⁸)ᵣR¹⁰, C₁₋₆ haloalkyl, and (CH₂)ᵣphenyl substituted with 0-3 R¹³;

m is 1-2;

n is 1-2;

q is 1-2; and

r is 0-2.

In still yet another embodiment, compounds of formula (I) are compounds in which:

R³ is independently selected from C₁-C₆ alkyl and C₃-C₆ cycloalkyl;

R⁴ is aryl, wherein the aryl may be optionally substituted with one or more substituents selected from C₁₋₈ alkyl, C₃₋₆ cycloalkyl, Cl, Br, I, F, NO₂, CN, C₁₋₆ haloalkyl and phenyl substituted with 0-3 R¹³;

R¹³, at each occurrence, is independently selected from C₁₋₆ alkyl, C₃₋₆ cycloalkyl, Cl, F, Br, I, CN, NO₂, OH, and SH;

T¹ and T² are independently selected from hydrogen, C₁₋₈ alkyl, C₃₋₆ cycloalkyl, Cl, Br, I, F, NO₂, and CN;

W¹, W² and W³ are independently selected from hydrogen, C₁₋₈ alkyl and C₃₋₆ cycloalkyl;

m is 1; and

n is 1.

In another embodiment, compounds of the present invention are selected from the compounds exemplified in Examples 1 to 123.

In another embodiment, the present invention relates to pharmaceutical compositions, comprising a pharmaceutically acceptable carrier and a therapeutically effective amount of a compound of the present invention.

In another embodiment, the present invention relates to a method for modulation of chemokine receptor activity comprising administering to a patient in need thereof a therapeutically effective amount of a compound of the present invention.

In another embodiment, the present invention provides a method for treating or preventing asthma, comprising administering to a patient in need thereof a therapeutically effective amount of a compound of the present invention.

In another embodiment, the modulation of chemokine receptor activity comprises contacting a CCR3 receptor with an effective inhibitory amount of a compound of the present invention.

In another embodiment, the present invention provides a method for treating or preventing inflammatory disorders comprising administering to a patient in need thereof a therapeutically effective amount of a compound of the present invention, or a pharmaceutically acceptable salt thereof.

In another embodiment, the present invention provides a method for treating or preventing disorders selected from asthma, allergic rhinitis, atopic dermatitis, inflammatory bowel diseases, idiopathic pulmonary fibrosis, bullous pemphigoid, helminthic parasitic infections, allergic colitis, eczema, conjunctivitis, transplantation, familial eosinophilia, eosinophilic cellulitis, eosinophilic pneumonias, eosinophilic fasciitis, eosinophilic gastroenteritis, drug induced eosinophilia, HIV infection, cystic fibrosis, Churg-Strauss syndrome, lymphoma, Hodgkin's disease, and colonic carcinoma, preferably asthma, allergic rhinitis, atopic dermatitis, and inflammatory bowel diseases, more preferably asthma.

In another embodiment, the present invention provides a pharmaceutical composition comprised of a pharmaceutical composition of the present invention and one or more active ingredients.

In another embodiment, the present invention provides a method for treating inflammatory disorders comprising administering to a patient in need thereof a therapeutically effective amount of a pharmaceutical composition of the present invention and one or more active ingredients.

It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

### Definitions

The compounds herein described may have asymmetric centers and/or exhibit polymorphism. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, polymorphic forms and all geometric isomeric forms of a structure are intended, unless the specific stereochemistry or isomeric form is specifically indicated.

The term “substituted,” as used herein, means that any one or more hydrogens on the designated atom or ring is replaced with a selection from the indicated group, provided that the designated atom's normal valency is not exceeded, and that the substitution results in a stable compound. When a substituent is keto (i.e., ═O), then 2 hydrogens on the atom are replaced.

When any variable (e.g., R^{a}) occurs more than one time in any constituent or formula for a compound, its definition at each occurrence is independent of its definition at every other occurrence. Thus, for example, if a group is shown to be substituted with 0-2 R^{a}, then said group may optionally be substituted with up to two R^{a} groups and R^{a} at each occurrence is selected independently from the definition of R^{a}. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “C₁₋₈ alkyl” is intended to include both branched and straight-chain saturated aliphatic hydrocarbon groups having the specified number of carbon atoms, examples of which include, but are not limited to, methyl, ethyl, n-propyl, i-propyl, n-butyl, i-butyl, sec-butyl, t-butyl, pentyl, and hexyl, C₁₋₆ alkyl, is intended to include C₁, C₂, C₃, C₄, C₅ and C₆ alkyl groups. “Alkenyl” is intended to include hydrocarbon chains of either a straight or branched configuration and one or more unsaturated carbon-carbon bonds which may occur in any stable point along the chain, such as ethenyl, propenyl, and the like. “Alkynyl” is intended to include hydrocarbon chains of either a straight or branched configuration and one or more unsaturated triple carbon-carbon bonds which may occur in any stable point along the chain, such as ethynyl, propynyl, and the like. “C₃₋₆ cycloalkyl” is intended to include saturated ring groups having the specified number of carbon atoms in the ring, including mono-, bi-, or poly-cyclic ring systems, such as cyclopropyl, cyclobutyl, cyclopentyl, cyclohexyl, and cycloheptyl in the case of C₇ cycloalkyl, C₃₋₆ cycloalkyl, is intended to include C₃, C₄, C₅, and C₆ cycloalkyl groups.

“Halo” or “halogen” as used herein refers to fluoro, chloro, bromo, and iodo; and “haloalkyl” is intended to include both branched and straight-chain saturated aliphatic hydrocarbon groups, for example CF₃, having the specified number of carbon atoms, substituted with 1 or more halogen (for example —CᵥF_{w} where v=1 to 3 and w=1 to (2v+1)).

As used herein, the term “heterocyclyl”, “heterocycle” or “heterocyclic system” is intended to mean a stable 5, 6, or 7-membered monocyclic or bicyclic or 7, 8, 9, or 10-membered bicyclic heterocyclic ring which is saturated, partially unsaturated or unsaturated (aromatic), and which consists of carbon atoms and 1, 2, 3, or 4 heteroatoms independently selected from the group consisting of N, NH, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized. It is preferred that when the total number of S and O atoms in the heterocycle exceeds 1, then these heteroatoms are not adjacent to one another. As used herein, the term “aromatic heterocyclic system” is intended to mean a stable 5- to 7-membered monocyclic or bicyclic or 7- to 10-membered bicyclic heterocyclic aromatic ring which consists of carbon atoms and from 1 to 4 heterotams independently selected from the group consisting of N, O and S.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, β-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, tetrazolyl, and xanthenyl. Heterocycles include, but are not limited to, pyridinyl, thiophenyl, furanyl, indazolyl, benzothiazolyl, benzimidazolyl, benzothiaphenyl, benzofuranyl, benzoxazolyl, benzisoxazolyl, quinolinyl, isoquinolinyl, imidazolyl, indolyl, isoidolyl, piperidinyl, piperidonyl, 4-piperidonyl, piperonyl, pyrazolyl, 1,2,4-triazolyl, 1,2,3-triazolyl, tetrazolyl, thiazolyl, oxazolyl, pyrazinyl, and pyrimidinyl. Also included are fused ring and spiro compounds containing, for example, the above heterocycles.

The compounds herein described may have asymmetric centers. While all enantiomers/diasteriomers are intended to be covered by the instant application, one enantiomer of a compound of Formula (I) may display superior biological activity over the opposite enantiomer. When required, separation of the racemic material can be achieved by methods known in the art. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers which release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

In addition, compounds of the formula I are, subsequent to their preparation, preferably isolated and purified to obtain a composition containing an amount by weight equal to or greater than 99% formula I compound (“substantially pure” compound I), which is then used or formulated as described herein. Such “substantially pure” compounds of the formula I are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. Only stable compounds are envisioned for this invention.

“Therapeutically effective amount” is intended to include an amount of a compound of the present invention alone or an amount of the combination of compounds claimed or an amount of a compound of the present invention in combination with other active ingredients effective to treat the inflammatory diseases described herein.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

### Synthesis

The compounds of Formula I can be prepared using the reactions and techniques described below. The reactions are performed in a solvent appropriate to the reagents and materials employed and suitable for the transformations being effected. It will be understood by those skilled in the art of organic synthesis that the functionality present on the molecule should be consistent with the transformations proposed. It will sometimes be desirable or necessary to modify the order of the synthetic steps or to select one particular process over another in order to obtain a desired compound of the invention, and such modifications will be recognized by those skilled in the art. It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is T. W. Greene and P. G. M. Wuts, Protective Groups In Organic Synthesis, Wiley and Sons, 1999. Some protecting groups are also discussed in M. Bodanszky and A. Bodanszky, The Practice of peptide Synthesis, 2nd ed., Springer-Verlag, 1994; and M. Bodanszky, Peptide Chemistry, 2nd ed., Springer-Verlag, 1993.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula I into another compound of formula I. Examples of such manipulations are conversion of an ester or a ketone to an alcohol; conversion of an ester to a ketone; interconversions of esters, acids, and amides; alkylation, acylation, and sulfonylation of alcohols and amines; and many others. Substituents can also be added using common reactions such as alkylation, acylation, halogenation, or oxidation. Such manipulations are well known in the art, and many reference works summarize procedures and methods for such manipulations. Some reference works which gives examples and references to the primary literature of organic synthesis for many functional group manipulations as well as other transformations commonly used in the art of organic synthesis are R. C. Larock, Comprehensive Organic Transformations, VCH, 1989; A. Katritzky et al. (series editors), Comprehensive Organic Functional Group Transformations, Pergamon, 1995; and B. Trost and I. Fleming (series editors), Comprehensive Organic Synthesis, Pergamon, 1991.

Generally, compounds in the scope of this patent application can be synthesized by solid-phase parallel synthesis via the route described in Scheme 1. The solid phase synthesis was performed on an aldehyde resin. Primary amines were attached to the resin by reductive amination and the final derivated amine products were cleaved by acid hydrolysis. Thus, amine 2 was loaded onto resin 1 using a reducing agent such as sodium triacetoxyborohydride or sodium cyanoborohydride. The resulting polymer-bound amine 3 was acylated by a N-protected natural or unnatural amino acid chloride 4 (X=Cl) in the presence of a base or an acid scavenger to yield protected amide 5. The coupling can be performed at −78° C. to room temperature. Coupling can also be done via the free carboxylic acid 4 (X=OH) using standard coupling reagents like EDC (N-ethyl,N-dimethylaminopropylcarbodiimide), HATU (O-(7-azabenzotriazol-1-yl)-1,1,3,3,-tetramethyluronium hexafluorophosphate) or PyBOP (benzotriazole-1-yl-oxy-tris-pyrrolidino-phosphonium hexaflurophosphate) in the presence of a tertiary amine base, such as triethylamine, N,N-diisopropylethylamine, or N-methylmorpholine. The protecting group of the nitrogen of 5 was subsequently removed to yield amine 6. Examples of protecting groups that can be used include, but are not limited to, phthalimide, which can be removed by a hydrazine, a reaction familiar to one skilled in the art; and Fmoc, which can be removed by piperidine by conditions familiar to one skilled in the art. Many of the Fmoc protected amino acids are commercially available or may be prepared from commercial amino acid derivatives by simple protecting group manipulations. Others may be synthesized in racemic form using the strecker synthesis or amidomalonate synthesis. In addition, the Myers pseudoephedrine glycinamide alkylation method (Myers, A. G.; Gleason, J. L.; Yoon, T.; Kung, D. W. J. Am. Chem. Soc. 1997, 119, 656-673), Schollkopf stereoselective alkylation (Schollkoft, U.; Hartwig, W.; Groth, U. Angew. Chem. Int. Ed. Engl. 1979, 18, 863), and Evans electrophilic azidation (Evans, D. A.; Britton, T. C.; Ellman, J. A.; Dorow, R. L. J. Am. Chem. Soc. 1990, 112, 4011) may be used to prepare unnatural amino acids in enantionmerically pure form.

The intermediate amine 6 was then coupled with N-protected piperidone 7 by reductive amination to provide secondary amine 8. Acylation of 8 with an acid or acid chloride 9 followed by removal of the protecting group on the piperidine nitrogen produced amine 10. Alkylation of 10 with aldehyde 11 by reductive amination followed by cleavage of product 12 from the polymer support generated compound 13.

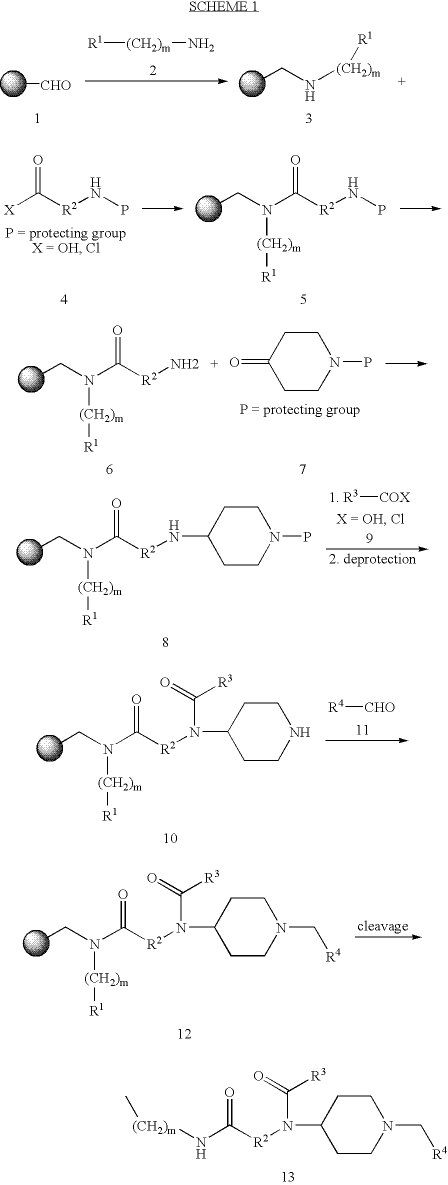

A representative method leading to a compound of the present invention is illustrated in Scheme 2. The polymer-bound 3,4-dimethoxybenzylamine 15 was obtained by loading 3,4-dimethoxybenzylamine 14 onto 4-(4-formyl-3-methoxyphenoxy)butyryl Nova Gel (FMP Resin) 1 using sodium triacetoxyborohydride in 2% acetic acid (AcOH) in methylene chloride (CH₂Cl₂). Amine 15 was then coupled to Fmoc-(4-aminomethyl)-benzoic acid 16 using HATU in the presence of N,N-diisopropylethylamine to give amide 17. Removal of the Fmoc group of 17 with 20% piperidine in DMF (N,N-dimethylformamide) provided amine 18. Amine 18 was coupled with Fmoc-4-piperidone 19 by reductive amination to yield amine 20. Acylation of amine 20 with 2-ethylbutyryl chloride 21 in the presence of DIEA (diethylamine) followed by removal of the Fmoc group generated amine 22. Alkylation of amine 22 by reductive amination with 3-hydroxy-4-methoxy benzaldehyde 23 followed by cleavage of product 24 from the polymer support with a 1:1 mixture of trifluoroacetic acid (TFA) and CH₂Cl₂ for 1 hour provided antagonist 25.

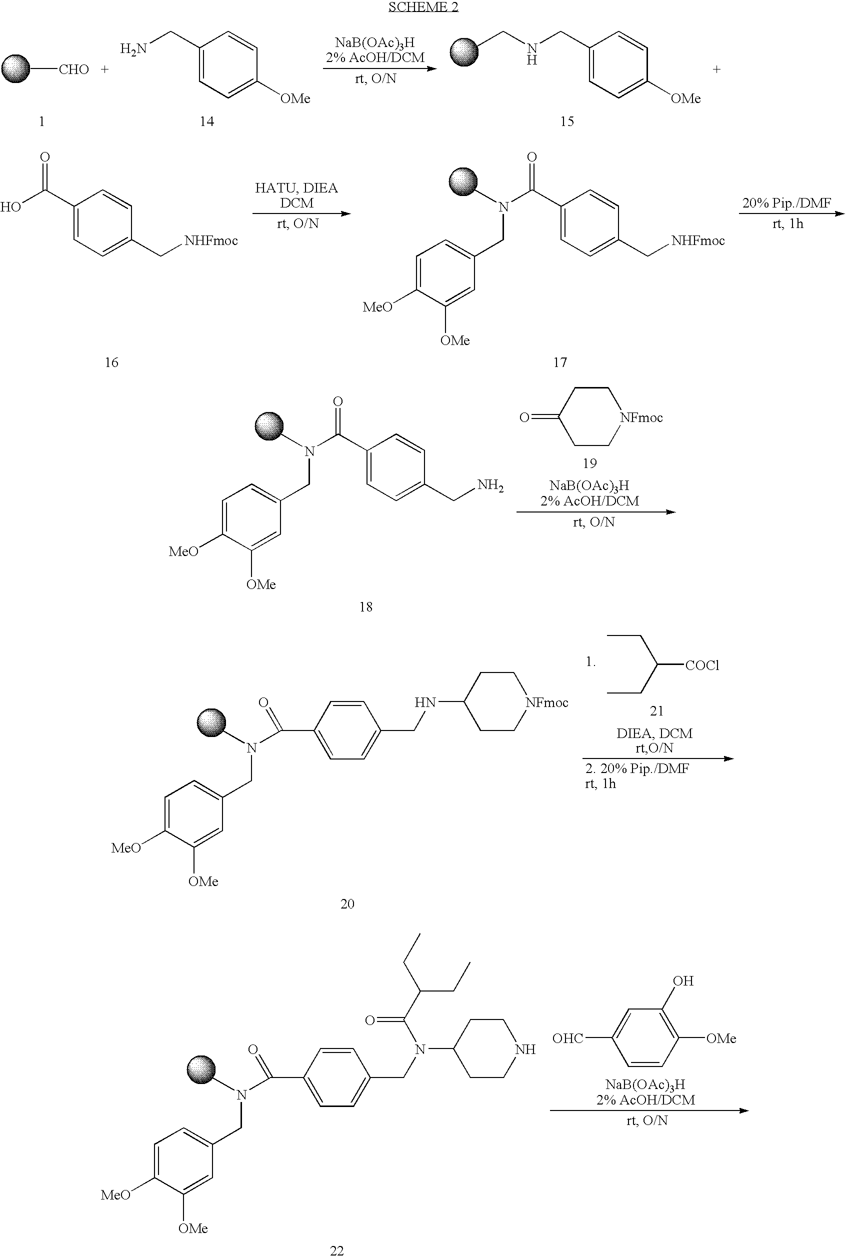

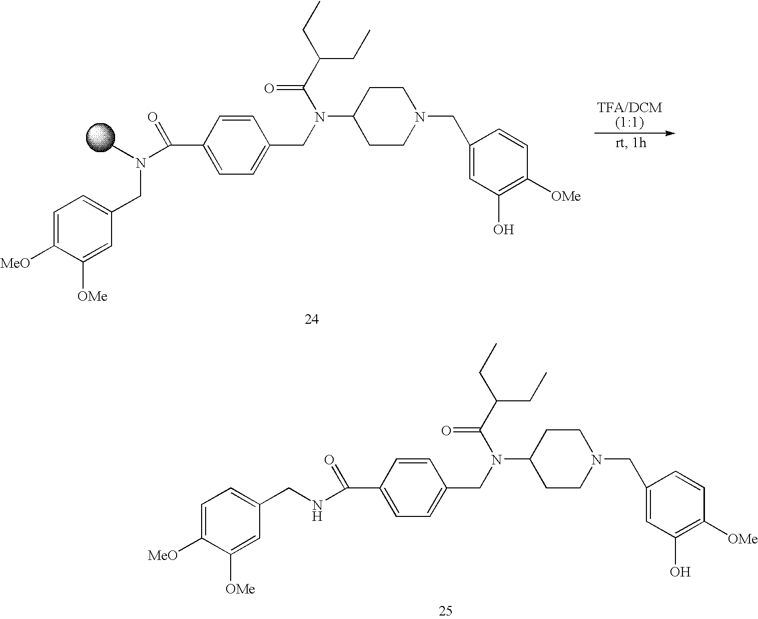

### EXAMPLES

The compounds of the present invention and their preparation can be understood further by the following working examples. These examples are meant to be illustrative of the present invention, and are not to be taken as limiting thereof.

Abbreviations used in the examples are defined as follows: “1 x” for once, “2×” for twice, “3×” for thrice, “° C.” for degrees Celsius, “rt” for room temperature, “eq” for equivalent, “mm” for millimeter, “min” for minute(s), “mmol” for millimole(s), “g” for gram(s), “mg” for milligram(s), “h” for hour(s), “mL” for milliliter(s), and “nm” for nanometers.

### Material and Instrument

The resin used for the synthesis was 4-(4-Formyl-3-methoxyphenoxy)butyryl NovaGel 1 (Nova-FMP resin, Novabiochem, loading of 0.55 mmol/g). Reactions were performed in bottom-and-top capped polypropylene-fritted tubes manufactured by Bio-Rad Laboratories. Reactions in the polypropylene tubes were shaken using Labquake Tube Rotor/Rocker manufactured by Thermolyne. High Performance Liquid Chromatography (HPLC) analyses were performed on a Hewlett-Packard 1090 liquid chromatography system using a photodiode array detector and ODS-A 5 mm (C18, 4.5 mm×50 mm) YMC slimbore column with a gradient of 0% acetonitrile/water containing 0.1% TFA to 100% acetonitrile over 8 min at 3 mL/min flow rate. Peak areas were integrated at 220 and 254 nm.

### Yields and Purities

Crude products cleaved from polymer support were purified by column chromatography on silica gel (2 g LC-Si column, Supelco, ethyl acetate/hexane to ethyl acetate/methanol gradient used). Generally 2-15 mg of product, with greater than 95% purity based on HPLC peak area at 220 nm, was obtained after purification.

### Example 1

### N-(3,4-Dimethoxybenzyl)-4-({(2-ethylbutyryl)-[1-(3-hydroxy-4-methoxybenzyl)piperidin-4-yl]amino}methyl)benzamide

### Step A

Nova-FMP resin 1 (1.0 g, 0.55 mmol/g) was mixed with 3,4-dimethoxy benzylamine (5 eq) and NaB(OAc)₃H (5 eq) in 20 mL CH₂Cl₂. The suspension was shaken at room temperature for 12 h. The resin was washed with DMF (3×), methanol (MeOH, 3×), and CH₂Cl₂ (3×), and then dried in vacuo to provide a resin.

### Step B

The resin (0.2 g, ca 0.1 mmol) from Step A, Example 1 was suspended in 5 mL CH₂Cl₂. Fmoc-(4-aminomethyl)-benzoic acid (5 eq), HATU (5 eq) and DIEA (10 eq) were added. The mixture was shaken at room temperature for 12 h. The polymer was filtered and then washed with DMF (3×), MeOH (3×) and CH₂Cl₂ (3×) to provide a resin.

### Step C

The resin from Step B, Example 1 was treated with 20% piperidine in DMF (5 mL) for 1 h at room temperature, and then washed with DMF (3×), MeOH (3 X), and CH₂Cl₂ (3×) to provide a resin.

### Step D

The resin from Step C, Example 1 (0.2 g, ca 0.1 mmol) was mixed with Fmoc-4-piperidone (4 eq) and NaB(OAc)₃H (4 eq) in 5 mL CH₂Cl₂. The resulting suspension was shaken at room temperature for 3 h. The polymer was filtered, washed with CH₂Cl₂ (3×) and treated again with Fmoc-4-piperidone (4 eq) and NaB(OAc)₃H (4 eq) in 20 mL CH₂Cl₂ for 2 h. At the conclusion of this period, the resin was filtered, washed with DMF (3×), MeOH (3×), and CH₂Cl₂ (3×), and then dried in vacuo to provide a resin.

### Step E

The resin from Step D, Example 1 (0.2 g, ca 0.1 mmol) was suspended in 5 mL CH₂Cl₂. 2-Ethylbutyryl chloride (5 eq) and DIEA (10 eq) were added, and the resulting mixture was shaken at room temperature for 12 h. At the conclusion of this period, the polymer was filtered, washed with DMF (3×), MeOH (3×), and CH₂Cl₂ (3×). The polymer was then Fmoc deprotected over a 60 minute period using 20% piperidine in DMF, and then washed with DMF (3×), MeOH (3×), and CH₂Cl₂ (3×) to provide a resin.

### Step F

The resin from Step E, Example 1 (0.1 g, ca 0.05 mmol) was mixed with 3-hydroxy-4-methoxy benzaldehyde (5 eq) and NaB(OAc)₃H (5 eq) in 5 mL CH₂Cl₂. The resulting suspension was shaken at room temperature for 12 h. After this time, the resin was washed with DMF (3×), MeOH (3×), and CH₂Cl₂ (3×) and then dried in vacuo to provide a resin.

### Step G

### Example 1

The resin from Step F, Example 1 (0.1 g, ca 0.05 mmol) was suspended in 1 mL of a 1:1 mixture of TFA and CH₂Cl₂ and shaken at room temperature for 1 h. At the conclusion of this period, the resin was washed with CH₂Cl₂ (2×1 mL). The combined filtrates were evaporated to dryness to provide a residue. The residue was purified by chromatography on silica gel (ethyl acetate/hexane (1:1) to ethyl acetate gradient was used) to provide Example 1 as a clolorless oil (5 mg). MS (EI) (M+H)⁺ 618.4.

Examples 2 to 123 were prepared by procedures analogous to those described for the preparation of Example 1.

### Example 2

### 4-({(2-Ethylbutyryl)-[1-(3-hydroxy-4-methoxybenzyl)piperidin-4-yl]amino}methyl)-N-(3-methylbenzyl)benzamide

MS (EI) (M+H)⁺ 572.3.

### Example 3

### 4-{[(2-Ethylbutyryl)-[1-(3-hydroxy-4-methoxybenzyl)piperidin-4-yl]amino}methyl)-N-(4-methoxybenzyl)benzamide

MS (EI) (M+H)⁺ 588.3.

### Example 4

### 4-({(2-Ethylbutyryl)-[1-(3-hydroxy-4-methoxybenzyl)piperidin-4-yl]amino}methyl)-N-(3-methoxybenzyl)benzamide

MS (EI) (M+H)⁺ 588.3.

### Example 5

### N-(3-Chlorobenzyl)-4-({(2-ethylbutyryl)-[1-(3-hydroxy-4-methoxybenzyl)piperidin-4-yl]amino}methyl)benzamide

MS (EI) (M+H)⁺ 592.3.

### Example 6

### 4-({(2-Ethylbutyryl)-[1-(3-hydroxy-4-methoxybenzyl)piperidin-4-yl]amino}methyl)-N-(2-fluorobenzyl)benzamide

MS (EI) (M+H)⁺ 576.3.

### Example 7

### 4-({(2-Ethylbutyryl)-[1-(3-hydroxy-4-methoxybenzyl)piperidin-4-yl]amino}methyl)-N-pyridin-3-ylmethylbenzamide

MS (EI) (M+H)⁺ 559.3.

### Example 8

### 4-({(2-Ethylbutyryl)-[1-(3-hydroxy-4-methoxybenzyl)piperidin-4-yl]amino}methyl)-N-(4-fluorobenzyl)benzamide

MS (EI) (M+H)⁺ 576.3.

### Example 9

### N-(3,4-Difluorobenzyl)-4-({(2-ethylbutyryl)-[1-(3-hydroxy-4-methoxybenzyl)piperidin-4-yl]amino}methyl)benzamide

MS (EI) (M+H)⁺ 594.3.

### Example 10

### N-(3-Bromobenzyl)-4-({(2-ethylbutyryl)-[1-(3-hydroxy-4-methoxybenzyl)piperidin-4-yl]amino}methyl)benzamide

MS (EI) (M+H)⁺ 636.2.

### Example 11

### N-Benzo[1,3]dioxol-5-ylmethyl-4-({(2-ethylbutyryl)-[1-(3-hydroxy-4-methoxybenzyl)piperidin-4-yl]amino}methyl)benzamide

MS (EI) (M+H)⁺ 602.3.

### Example 12

### 4-({(2-Ethylbutyryl)-[1-(3-hydroxy-4-methoxybenzyl)piperidin-4-yl]amino}methyl)-N-(3-nitrobenzyl)benzamide

MS (EI) (M+H)⁺ 603.3.

### Example 13

### N-(4-Bromobenzyl)-4-({(2-ethylbutyryl)-[1-(3-hydroxy-4-methoxybenzyl)piperidin-4-yl]amino}methyl)benzamide

MS (EI) (M+H)⁺ 636.2.

### Example 14

### 4-({[1-(4-Fluorobenzyl)piperidin-4-yl]isobutyrylamino}methyl)-N-(4-methoxybenzyl)benzamide

MS (EI) (M+H)⁺ 532.3.

### Example 15

### 4-({Cyclobutanecarbonyl-[1-(4-fluorobenzyl)piperidin-4-yl]amino}methyl)-N-(4-methoxybenzyl)benzamide

MS (EI) (M+H)⁺ 544.3.

### Example 16

### 4-({Butyryl-[1-(4-fluorobenzyl)piperidin-4-yl]amino}methyl)-N-(4-methoxybenzyl)benzamide

MS (EI) (M+H)⁺ 532.3.

### Example 17

### 4-({Cyclopropanecarbonyl-[1-(4-fluorobenzyl)piperidin-4-yl]amino}methyl)-N-(4-methoxybenzyl)benzamide

MS (EI) (M+H)⁺ 530.3.

### Example 18

### 4-({(3,3-Dimethylbutyryl)-[1-(4-fluorobenzyl)piperidin-4-yl]amino}methyl)-N-(4-methoxybenzyl)benzamide

MS (EI) (M+H)⁺ 560.3.

### Example 19

### 4-{[[1-(4-Fluorobenzyl)piperidin-4-yl]-(3-methylbutyryl)amino]methyl}-N-(4-methoxybenzyl)benzamide

MS (EI) (M+H)⁺ 546.3.

### Example 20

### Furan-2-carboxylic acid [1-(4-fluorobenzyl)piperidin-4-yl]-[4-(4-methoxy-benzylcarbamoyl)benzyl]amide

MS (EI) (M+H)⁺ 556.3.

### Example 21

### 4-({(3,3-Dimethylbutyryl)-[1-(3-hydroxy-4-methoxybenzyl)piperidin-4-yl]amino}methyl)-N-(4-methoxybenzyl)benzamide

MS (EI) (M+H)⁺ 588.3.

### Example 22

### 4-({[1-(3-Hydroxy-4-methoxybenzyl)piperidin-4-yl]isobutyrylamino}methyl)-N-(4-methoxybenzyl)benzamide

MS (EI) (M+H)⁺ 560.3.

### Example 23

### 4-({[1-(4-Fluorobenzyl)piperidin-4-yl]propionylamino}methyl)-N-(4-methoxybenzyl)benzamide

MS (EI) (M+H)⁺ 518.3.

### Example 24

### 4-({(2-Cyclopentylacetyl)-[1-(4-fluorobenzyl)piperidin-4-yl]amino}methyl)-N-(4-methoxybenzyl)benzamide

MS (EI) (M+H)⁺ 572.3.

### Example 25

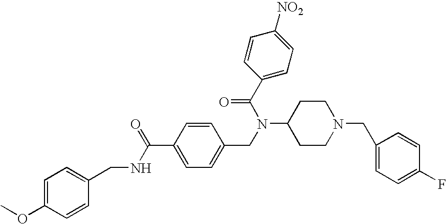

### 4-Nitrobenzoic acid [1-(4-fluorobenzyl)piperidin-4-yl]-[4-(4-methoxybenzylcarbamoyl)benzyl]amide

MS (EI) (M+H)⁺ 611.3.

### Example 26

### Benzo[1,3]dioxole-5-carboxylic acid [1-(4-fluorobenzyl)piperidin-4-yl]-[4-(4-methoxybenzylcarbamoyl)benzyl]amide

MS (EI) (M+H)⁺ 610.3.

### Example 27

### 4-({Cyclobutanecarbonyl-[1-(3-hydroxy-4-methoxybenzyl)piperidin-4-yl]amino}methyl)-N-(4-methoxybenzyl)benzamide

MS (EI) (M+H)⁺ 572.3.

### Example 28

### 4-{[[1-(4-Fluorobenzyl)piperidin-4-yl]-(1-methyl-cyclohexanecarbonyl)amino]methyl}-N-(4-methoxybenzyl)benzamide

MS (EI) (M+H)⁺ 586.3.

### Example 29

### 4-({Cyclopropanecarbonyl-[1-(3-hydroxy-4-methoxybenzyl)piperidin-4-yl]amino}methyl)-N-(4-methoxybenzyl)benzamide

MS (EI) (M+H)⁺ 558.3.

### Example 30

### 4-({(3-Cyclopentylpropionyl)-[1-(4-fluorobenzyl)piperidin-4-yl]amino}methyl)-N-(4-methoxybenzyl)benzamide

MS (EI) (M+H)⁺ 586.3.

### Example 31

### 4-{[[1-(4-Fluorobenzyl)piperidin-4-yl]-(2-methylbutyryl)amino]methyl}-N-(4-methoxybenzyl)benzamide

MS (EI) (M+H)⁺ 546.3.

### Example 32

### N-[1-(4-Fluorobenzyl)piperidin-4-yl]-N-[4-(4-methoxy-benzylcarbamoyl)benzyl]nicotinamide

MS (EI) (M+H)⁺ 567.3.

### Example 33

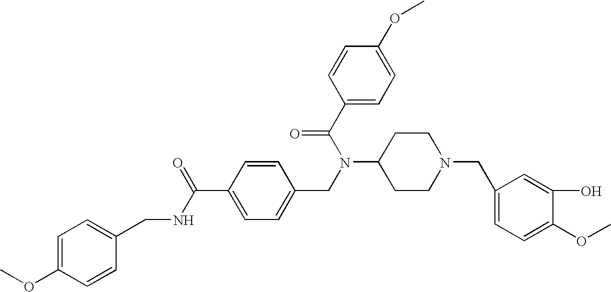

### 4-Methoxybenzoic acid [1-(3-hydroxy-4-methoxybenzyl)piperidin-4-yl]-[4-(4-methoxybenzylcarbamoyl)benzyl]amide

MS (EI) (M+H)⁺ 624.3.

### Example 34

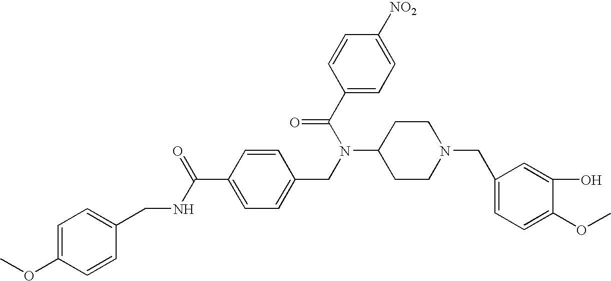

### 4-Methoxybenzoic acid [1-(3-hydroxy-4-methoxybenzyl)piperidin-4-yl]-[4-(4-methoxybenzylcarbamoyl)benzyl]amide

MS (EI) (M+H)⁺ 639.3.

### Example 35

### 4-{[[1-(4-Chlorobenzyl)-piperidin-4-yl]-(2-ethylbutyryl)amino]methyl}-N-(4-methoxybenzyl)benzamide

MS (EI) (M+H)⁺ 576.3.

### Example 36

### 4-({(2-Ethylbutyryl)-[1-(4-fluorobenzyl)piperidin-4-yl]amino}methyl)-N-(4-methoxybenzyl)benzamide

MS (EI) (M+H)⁺ 560.3.

### Example 37

### 4-{[[1-(4-Bromobenzyl)piperidin-4-yl]-(2-ethylbutyryl)amino]methyl}-N-(4-methoxybenzyl)benzamide

MS (EI) (M+H)⁺ 620.2.

### Example 38

### 4-{[[1-(4-Cyanobenzyl)piperidin-4-yl]-(2-ethylbutyryl)amino]methyl}-N-(4-methoxybenzyl)benzamide

MS (EI) (M+H)⁺ 567.3.

### Example 39

### 4-({(2-Ethylbutyryl)-[1-(4-nitrobenzyl)piperidin-4-yl]amino}methyl)-N-(4-methoxybenzyl)benzamide

MS (EI) (M+H)⁺ 587.3.

### Example 40

### 4-({(2-Ethylbutyryl)-[1-(3-hydroxybenzyl)piperidin-4-yl]amino}methyl)-N-(4-methoxybenzyl)benzamide

MS (EI) (M+H)⁺ 558.3.

### Example 41

### 4-({(2-Ethylbutyryl)-[1-(4-methoxybenzyl)piperidin-4-yl]amino}methyl)-N-(4-methoxybenzyl)benzamide

MS (EI) (M+H)⁺ 572.3.

### Example 42

### 4-({(2-Ethylbutyryl)-[1-(4-methylbenzyl)piperidin-4-yl]amino}methyl)-N-(4-methoxybenzyl)benzamide

MS (EI) (M+H)⁺ 556.4.

### Example 43

### 4-{[[1-(2,4-Difluorobenzyl)piperidin-4-yl]-(2-ethylbutyryl)amino]methyl}-N-(4-methoxybenzyl)benzamide

MS (EI) (M+H)⁺ 578.3.

### Example 44

### 4-({(2-Ethylbutyryl)-[1-(1H-indol-3-ylmethyl)piperidin-4-yl]amino}methyl)-N-(4-methoxybenzyl)benzamide

MS (EI) (M+H)⁺ 581.3.

### Example 45

### 4-({(2-Ethylbutyryl)-[1-(3-methylbenzyl)piperidin-4-yl]amino}methyl)-N-(4-methoxybenzyl)benzamide

MS (EI) (M+H)⁺ 556.4.

### Example 46

### 4-{[[1-(3-Bromobenzyl)piperidin-4-yl]-(2-ethylbutyryl)amino]methyl}-N-(4-methoxybenzyl)benzamide

MS (EI) (M+H)⁺ 620.2.

### Example 47

### 4-({(2-Ethylbutyryl)-[1-(4-methylsulfanylbenzyl)piperidin-4-yl]amino}methyl)-N-(4-methoxybenzyl)benzamide

MS (EI) (M+H)⁺ 588.3.

### Example 48

### 4-({[1-(4-Fluorobenzyl)piperidin-4-yl]isobutyrylamino}methyl)-N-(3-methoxybenzyl)benzamide

MS (EI) (M+H)⁺ 532.3.

### Example 49

### 4-{[[1-(4-Chlorobenzyl)piperidin-4-yl]-(2-ethylbutyryl)amino]methyl}-N-(3-methoxybenzyl)benzamide

MS (EI) (M+H)⁺ 576.3.

### Example 50

### 4-{[[1-(4-Chlorobenzyl)piperidin-4-yl]-(2-ethylbutyryl)amino]methyl}-N-(3-methylbenzyl)benzamide

MS (EI) (M+H)⁺ 560.3.

### Example 51

### 4-{[[1-(4-Chlorobenzyl)piperidin-4-yl]-(2-ethylbutyryl)amino]methyl}-N-(3-fluorobenzyl)benzamide

MS (EI) (M+H)⁺ 564.3.

### Example 52

### 4-({(2-Ethylbutyryl)-[1-(4-fluorobenzyl)piperidin-4-yl]amino}methyl)-N-(3-methylbenzyl)benzamide

MS (EI) (M+H)⁺ 544.3.

### Example 53

### 4-{[[1-(4-Bromobenzyl)piperidin-4-yl]-(2-ethylbutyryl)amino]methyl}-N-(3-methylbenzyl)benzamide

MS (EI) (M+H)⁺ 604.3.

### Example 54

### 4-({(2-Ethylbutyryl)-[1-(4-fluorobenzyl)piperidin-4-yl]amino}methyl)-N-(3-fluorobenzyl)benzamide

MS (EI) (M+H)⁺ 548.3.

### Example 55

### 4-{[[1-(4-Bromobenzyl)piperidin-4-yl]-(2-ethylbutyryl)amino]methyl}-N-(3-methoxybenzyl)benzamide

MS (EI) (M+H)⁺ 620.2.

### Example 56

### N-(4-Chlorobenzyl)-4-({(2-ethylbutyryl)-[1-(4-fluorobenzyl)piperidin-4-yl]amino}methyl)benzamide

MS (EI) (M+H)⁺ 564.3.

### Example 57

### 4-({(2-Ethylbutyryl)-[1-(4-fluorobenzyl)piperidin-4-yl]amino}methyl)-N-(4-fluorobenzyl)benzamide

MS (EI) (M+H)⁺ 548.3.

### Example 58

### 4-({(2-Ethylbutyryl)-[1-(4-fluorobenzyl)piperidin-4-yl]amino}methyl)-N-(3-methoxybenzyl)benzamide

MS (EI) (M+H)⁺ 560.3.

### Example 59

### 4-({Isobutyryl-[1-(4-nitrobenzyl)piperidin-4-yl]amino}methyl)-N-(4-methoxybenzyl)benzamide

MS (EI) (M+H)⁺ 559.3.

### Example 60

### N-Benzyl-4-{[[1-(4-bromobenzyl)piperidin-4-yl]-(2-ethylbutyryl)amino]methyl}benzamide

MS (EI) (M+H)⁺ 590.2.

### Example 61

### N-(4-Chlorobenzyl)-4-({[1-(3-hydroxy-4-methoxybenzyl)piperidin-4-yl]isobutyrylamino}methyl)benzamide

MS (EI) (M+H)⁺ 564.3.

### Example 62

### N-Benzyl-4-({(2-ethylbutyryl)-[1-(4-fluorobenzyl)piperidin-4-yl]amino}methyl)benzamide

MS (EI) (M+H)⁺ 530.3.

### Example 63

### 4-{[[1-(4-Chlorobenzyl)piperidin-4-yl]-(2-ethylbutyryl)amino]methyl}-N-(4-fluorobenzyl)benzamide

MS (EI) (M+H)⁺ 564.3.

### Example 64

### 4-({[1-(4-Cyanobenzyl)piperidin-4-yl]isobutyrylamino}methyl)-N-(3-fluorobenzyl)benzamide

MS (EI) (M+H)⁺ 527.3.

### Example 65

### 4-{[[1-(4-Bromobenzyl)piperidin-4-yl]-(2-ethylbutyryl)amino]methyl}-N-(4-fluorobenzyl)benzamide

MS (EI) (M+H)⁺ 608.2.

### Example 66

### N-Benzyl-4-{([[1-(4-chlorobenzyl)piperidin-4-yl]-(2-ethylbutyryl)amino]methyl}benzamide

MS (EI) (M+H)⁺ 546.3.

### Example 67

### 4-({(2-Ethylbutyryl)-[1-(4-fluorobenzyl)piperidin-4-yl]amino}methyl)-N-(4-methylbenzyl)benzamide

MS (EI) (M+H)⁺ 544.3.

### Example 68

### N-Benzo[1,3]dioxol-5-ylmethyl-4-({(2-ethylbutyryl)-[1-(4-fluorobenzyl)piperidin-4-yl]amino}methyl)benzamide

MS (EI) (M+H)⁺ 574.3.

### Example 69

### N-Benzo[1,3]dioxol-5-ylmethyl-4-{[[1-(4-chlorobenzyl)piperidin-4-yl]-(2-ethylbutyryl)amino]methyl}benzamide

MS (EI) (M+H)⁺ 590.3.

### Example 70

### N-Benzyl-4-{[[1-(4-chlorobenzyl)piperidin-4-yl]-(2-ethylbutyryl)amino]methyl}benzamide

MS (EI) (M+H)⁺ 546.3.

### Example 71

### 4-({[1-(4-Chlorobenzyl)piperidin-4-yl]isobutyrylamino}methyl)-N-(3,4-dimethoxybenzyl)benzamide

MS (EI) (M+H)⁺ 578.3.

### Example 72

### 4-{[[1-(4-Chlorobenzyl)piperidin-4-yl]-(2-ethylbutyryl)amino]methyl}-N-(3,4-dimethoxybenzyl)benzamide

MS (EI) (M+H)⁺ 606.3.

### Example 73

### N-(3,4-Dimethoxybenzyl)-4-({(2-ethylbutyryl)-[1-(4-fluorobenzyl)piperidin-4-yl]amino}methyl)benzamide

MS (EI) (M+H)⁺ 590.3.

### Example 74

### N-(3,4-Dimethoxybenzyl)-4-({[1-(4-fluorobenzyl)piperidin-4-yl]isobutyrylamino}methyl)benzamide

MS (EI) (M+H)⁺ 562.3.

### Example 75

### 4-{[[1-(4-Chlorobenzyl)piperidin-4-yl]-(2,2-dimethylpropionyl)amino]methyl}-N-(3,4-dimethoxybenzyl)benzamide

MS (EI) (M+H)⁺ 592.3.

### Example 76

### 4-({[1-(4-Chlorobenzyl)piperidin-4-yl]propionylamino}methyl)-N-(3,4-dimethoxybenzyl)benzamide

MS (EI) (M+H)⁺ 564.3.

### Example 77

### 4-({[1-(4-Chlorobenzyl)piperidin-4-yl]cyclobutanecarbonylamino}methyl)-N-(3,4-dimethoxybenzyl)benzamide

MS (EI) (M+H)⁺ 590.3.

### Example 78

### 4-({[1-(4-Chlorobenzyl)piperidin-4-yl]cyclopentanecarbonylamino}methyl)-N-(3,4-dimethoxybenzyl)benzamide

MS (EI) (M+H)⁺ 604.3.

### Example 79

### 4-({Cyclobutanecarbonyl-[1-(4-fluorobenzyl)piperidin-4-yl]amino}methyl)-N-(3,4-dimethoxybenzyl)benzamide

MS (EI) (M+H)⁺ 574.3.

### Example 80

### 4-({Cyclopropanecarbonyl-[1-(4-fluorobenzyl)piperidin-4-yl]amino}methyl)-N-(3,4-dimethoxybenzyl)benzamide

MS (EI) (M+H)⁺ 560.3.

### Example 81

### N-(3,4-Dimethoxybenzyl)-4-({[1-(4-fluorobenzyl)piperidin-4-yl]propionylamino}methyl)benzamide

MS (EI) (M+H)⁺ 548.3.

### Example 82

### 4-({[1-(4-Chlorobenzyl)piperidin-4-yl]cyclopropanecarbonylamino}methyl)-N-(3,4-dimethoxybenzyl)benzamide

MS (EI) (M+H)⁺ 576.3.

### Example 83

### 4-({Cyclopentanecarbonyl-[1-(4-fluorobenzyl)piperidin-4-yl]amino}methyl)-N-(3,4-dimethoxybenzyl)benzamide

MS (EI) (M+H)⁺ 588.3.

### Example 84

### N-(3,4-Dimethoxybenzyl)-4-({(2,2-dimethylpropionyl)-[1-(4-fluorobenzyl)piperidin-4-yl]amino}methyl)benzamide

MS (EI) (M+H)⁺ 576.3.

### Example 85

### 4-({[1-(4-Bromobenzyl)piperidin-4-yl]isobutyrylamino}methyl)-N-(3,4-dimethoxybenzyl)benzamide

MS (EI) (M+H)⁺ 622.2.

### Example 86

### 4-{[[1-(4-Bromobenzyl)piperidin-4-yl]-(2-ethylbutyryl)amino]methyl}-N-(3,4-dimethoxybenzyl)benzamide

MS (EI) (M+H)⁺ 650.3.

### Example 87

### 4-({Butyryl-[1-(4-chlorobenzyl)piperidin-4-yl]amino}methyl)-N-(3,4-dimethoxybenzyl)benzamide

MS (EI) (M+H)⁺ 578.3.

### Example 88

### 4-({Butyryl-[1-(4-fluorobenzyl)piperidin-4-yl]amino}methyl)-N-(3,4-dimethoxybenzyl)benzamide

MS (EI) (M+H)⁺ 562.3.

### Example 89

### 4-({[1-(4-Cyanobenzyl)piperidin-4-yl]isobutyrylamino}methyl)-N-(3,4-dimethoxybenzyl)benzamide

MS (EI) (M+H)⁺ 569.3.

### Example 90

### 4-{[[1-(4-Cyanobenzyl)piperidin-4-yl]-(2-ethylbutyryl)amino]methyl}-N-(3,4-dimethoxybenzyl)benzamide

MS (EI) (M+H)⁺ 597.3.

### Example 91

### 4-({Cyclobutanecarbonyl-[1-(4-methylbenzyl)piperidin-4-yl]amino}methyl)-N-(3,4-dimethoxybenzyl)benzamide

MS (EI) (M+H)⁺ 570.3.

### Example 92

### 4-{[[1-(4-Bromobenzyl)piperidin-4-yl]-(2,2-dimethylpropionyl)amino]methyl}-N-(3,4-dimethoxybenzyl)benzamide

MS (EI) (M+H)⁺ 636.2.

### Example 93

### 4-({[1-(4-Bromobenzyl)piperidin-4-yl]cyclopentanecarbonylamino}methyl)-N-(3,4-dimethoxybenzyl)benzamide

MS (EI) (M+H)⁺ 648.2.

### Example 94

### 4-({[1-(4-Bromobenzyl)piperidin-4-yl]cyclobutanecarbonyl-amino}methyl)-N-(3,4-dimethoxybenzyl)benzamide

MS (EI) (M+H)⁺ 634.2.

### Example 95

### N-(3,4-Dimethoxybenzyl)-4-({[1-(3-hydroxy-4-methoxybenzyl)piperidin-4-yl]isobutyrylamino}methyl)benzamide

MS (EI) (M+H)⁺ 590.3.

### Example 96

### 4-({[1-(4-Cyanobenzyl)piperidin-4-yl]cyclopropanecarbonylamino}methyl)-N-(3,4-dimethoxybenzyl)benzamide

MS (EI) (M+H)⁺ 567.3.

### Example 97

### 4-({Butyryl-[1-(4-cyanobenzyl)piperidin-4-yl]amino}methyl)-N-(3,4-dimethoxybenzyl)benzamide

MS (EI) (M+H)⁺ 569.3.

### Example 98

### 4-({[1-(4-Bromobenzyl)piperidin-4-yl]butyrylamino}methyl)-N-(3,4-dimethoxybenzyl)benzamide

MS (EI) (M+H)⁺ 622.2.

### Example 99

### 4-({[1-(4-Bromobenzyl)piperidin-4-yl]cyclopropanecarbonylamino}methyl)-N-(3,4-dimethoxybenzyl)benzamide

MS (EI) (M+H)⁺ 620.2.

### Example 100

### N-(3,4-Dimethoxybenzyl)-4-({(2-ethylbutyryl)-[1-(3-hydroxy-4-methoxybenzyl)piperidin-4-yl]amino}methyl)benzamide

MS (EI) (M+H)⁺ 618.4.

### Example 101

### N-(3,4-Dimethoxybenzyl)-4-({(2-ethylbutyryl)-[1-(4-nitrobenzyl)piperidin-4-yl]amino}methyl)benzamide

MS (EI) (M+H)⁺ 617.3.

### Example 102

### N-(3,4-Dimethoxybenzyl)-4-({(2,2-dimethylpropionyl)-[1-(3-hydroxy-4-methoxybenzyl)piperidin-4-yl]amino}methyl)benzamide

MS (EI) (M+H)⁺ 604.3.

### Example 103

### N-(3,4-Dimethoxybenzyl)-4-({isobutyryl-[1-(4-methylbenzyl)piperidin-4-yl]amino}methyl)benzamide

MS (EI) (M+H)⁺ 558.3.

### Example 104

### N-(3,4-Dimethoxybenzyl)-4-({(2-ethylbutyryl)-[1-(4-methoxybenzyl)piperidin-4-yl]amino}methyl)benzamide

MS (EI) (M+H)⁺ 602.4.

### Example 105

### 4-{[[1-(4-Cyanobenzyl)piperidin-4-yl]-(2,2-dimethylpropionyl)amino]methyl}-N-(3,4-dimethoxybenzyl)benzamide

MS (EI) (M+H)⁺ 583.3.

### Example 106

### 4-({Cyclobutanecarbonyl-[1-(3-hydroxy-4-methoxybenzyl)piperidin-4-yl]amino}methyl)-N-(3,4-dimethoxybenzyl)benzamide

MS (EI) (M+H)⁺ 602.3.

### Example 107

### 4-({[1-(4-Cyanobenzyl)piperidin-4-yl]cyclobutanecarbonylamino}methyl)-N-(3,4-dimethoxybenzyl)benzamide

MS (EI) (M+H)⁺ 581.3.

### Example 108

### 4-({[1-(4-Cyanobenzyl)piperidin-4-yl]propionylamino}methyl)-N-(3,4-dimethoxybenzyl)benzamide

MS (EI) (M+H)⁺ 555.3.

### Example 109

### 4-({[1-(2,4-Difluorobenzyl)piperidin-4-yl]isobutyrylamino}methyl)-N-(3,4-dimethoxybenzyl)benzamide

MS (EI) (M+H)⁺ 580.3.

### Example 110

### N-(3,4-Dimethoxybenzyl)-4-({isobutyryl-[1-(4-methoxybenzyl)piperidin-4-yl]amino}methyl)benzamide

MS (EI) (M+H)⁺ 574.3.

### Example 111

### N-(3,4-Dimethoxybenzyl)-4-({isobutyryl-[1-(4-nitrobenzyl)piperidin-4-yl]amino}methyl)benzamide

MS (EI) (M+H)⁺ 589.3.

### Example 112

### N-(3,4-Dimethoxybenzyl)-4-({(2-ethylbutyryl)-[1-(4-methylbenzyl)piperidin-4-yl]amino}methyl)benzamide

MS (EI) (M+H)⁺ 586.4.

### Example 113

### 4-({Cyclopropanecarbonyl-[1-(4-nitrobenzyl)piperidin-4-yl]amino}methyl)-N-(3,4-dimethoxybenzyl)benzamide

MS (EI) (M+H)⁺ 587.3.

### Example 114

### 4-({[1-(4-Cyanobenzyl)piperidin-4-yl]cyclopentanecarbonylamino}methyl)-N-(3,4-dimethoxybenzyl)benzamide

MS (EI) (M+H)⁺ 595.3.

### Example 115

### 4-({Butyryl-[1-(4-nitrobenzyl)piperidin-4-yl]amino}methyl)-N-(3,4-dimethoxybenzyl)benzamide

MS (EI) (M+H)⁺ 589.3.

### Example 116

### N-Benzo[1,3]dioxol-5-ylmethyl-3-({(2-ethylbutyryl)-[1-(4-fluorobenzyl)piperidin-4-yl]amino}methyl)benzamide

MS (EI) (M+H)⁺ 574.3.

### Example 117

### N-Benzo[1,3]dioxol-5-ylmethyl-3-{[[1-(4-chlorobenzyl)piperidin-4-yl]-(2-ethylbutyryl)amino]methyl}benzamide

MS (EI) (M+H)⁺ 590.3.

### Example 118

### N-Benzyl-3-{[[1-(4-chlorobenzyl)piperidin-4-yl](2-ethylbutyryl)amino]methyl}benzamide

MS (EI) (M+H)⁺ 546.3.

### Example 119

### N-Benzyl-3-({(2-ethylbutyryl)-[1-(4-fluorobenzyl)piperidin-4-yl]amino}methyl)benzamide

MS (EI) (M+H)⁺ 530.3.

### Example 120

### 3-[[1-(1-Acetyl-1H-indol-3-ylmethyl)piperidin-4-yl]-(2-cyclopentylacetyl)amino]-N-[2-(4-chlorophenyl)ethyl]propionamide

MS (EI) (M+H)⁺ 591.3.

### Example 121

### N-[2-(4-Chlorophenyl)ethyl]-3-{(2-cyclopentylacetyl)-[1-(1-methyl-1H-indol-3-ylmethyl)piperidin-4-yl]amino}propionamide

MS (EI) (M+H)⁺ 563.3.

### Example 122

### N-[2-(4-Chlorophenyl)ethyl]-3-{(2-cyclopentylacetyl)-[1-(3-ethoxy-4-hydroxybenzyl)piperidin-4-yl]amino}propionamide

MS (EI) (M+H)⁺ 570.3.

### Example 123

### N-[2-(4-Chlorophenyl)ethyl]-3-{(2-cyclopentylacetyl)-[1-(1H-indol-3-ylmethyl)piperidin-4-yl]amino}propionamide

MS (EI) (M+H)⁺ 549.3.

Representative compounds prepared by the methods disclosed above are listed in Table 1.

TABLE 1

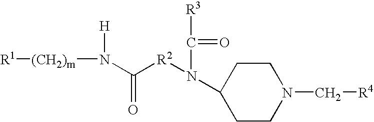

| Example | m | R¹ | R² | R³ | R⁴ | MS(M + 1)+ |
|---|---|---|---|---|---|---|
| 1 | 1 | 3,4-di-OMe-phenyl | 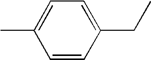 | 3-pentyl | 3-OH, 4-OMe-phenyl | 618.4 |
| 2 | 1 | 3-Me-phenyl | 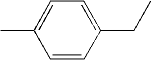 | 3-pentyl | 3-OH, 4-OMe-phenyl | 572.3 |
| 3 | 1 | 4-OMe-phenyl | 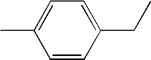 | 3-pentyl | 3-OH, 4-OMe-phenyl | 588.3 |
| 4 | 1 | 3-OMe-phenyl | 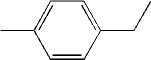 | 3-pentyl | 3-OH, 4-OMe-phenyl | 588.3 |
| 5 | 1 | 3-Cl-phenyl | 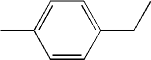 | 3-pentyl | 3-OH, 4-OMe-phenyl | 592.3 |
| 6 | 1 | 2-F-phenyl | 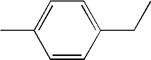 | 3-pentyl | 3-OH, 4-OMe-phenyl | 576.3 |
| 7 | 1 | 3-pyridyl | 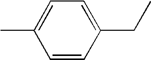 | 3-pentyl | 3-OH, 4-OMe-phenyl | 559.3 |
| 8 | 1 | 4-F-phenyl | 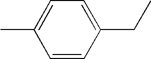 | 3-pentyl | 3-OH, 4-OMe-phenyl | 576.3 |
| 9 | 1 | 3,4-di-F-phenyl | 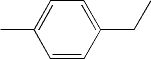 | 3-pentyl | 3-OH, 4-OMe-phenyl | 594.3 |
| 10 | 1 | 3-Br-phenyl | 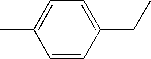 | 3-pentyl | 3-OH, 4-OMe-phenyl | 636.2 |
| 11 | 1 | 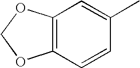 | 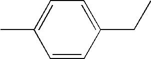 | 3-pentyl | 3-OH, 4-OMe-phenyl | 602.3 |
| 12 | 1 | 3-NO₂-phenyl | 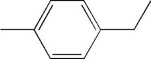 | 3-pentyl | 3-OH, 4-OMe-phenyl | 603.3 |
| 13 | 1 | 4-Br-phenyl | 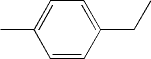 | 3-pentyl | 3-OH, 4-OMe-phenyl | 636.2 |
| 14 | 1 | 4-OMe-phenyl | 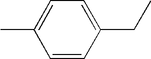 | i-propyl | 4-F-phenyl | 532.3 |
| 15 | 1 | 4-OMe-phenyl | 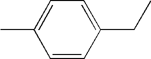 | cyclobutyl | 4-F-phenyl | 544.3 |
| 16 | 1 | 4-OMe-phenyl | 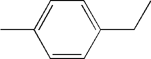 | n-propyl | 4-F-phenyl | 532.3 |
| 17 | 1 | 4-OMe-phenyl | 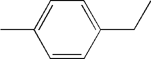 | cyclopropyl | 4-F-phenyl | 530.3 |
| 18 | 1 | 4-OMe-phenyl | 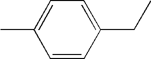 | t-butylmethyl | 4-F-phenyl | 560.3 |
| 19 | 1 | 4-OMe-phenyl | 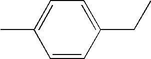 | i-butyl | 4-F-phenyl | 546.3 |
| 20 | 1 | 4-OMe-phenyl | 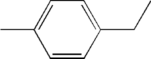 | 2-furanyl | 4-F-phenyl | 556.3 |
| 21 | 1 | 4-OMe-phenyl | 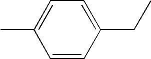 | t-butylmethyl | 3-OH, 4-Ome-phenyl | 588.3 |
| 22 | 1 | 4-OMe-phenyl | 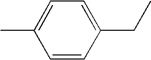 | i-propyl | 3-OH, 4-Ome-phenyl | 560.3 |
| 23 | 1 | 4-OMe-phenyl | 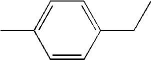 | ethyl | 4-F-phenyl | 518.3 |
| 24 | 1 | 4-OMe-phenyl | 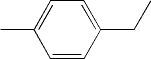 | cyclopentylmethyl | 4-F-phenyl | 572.3 |
| 25 | 1 | 4-OMe-phenyl | 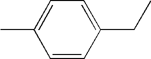 | 4-nitrophenyl | 4-F-phenyl | 611.3 |
| 26 | 1 | 4-OMe-phenyl | 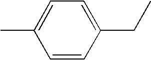 | 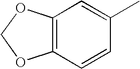 | 4-F-phenyl | 610.3 |
| 27 | 1 | 4-OMe-phenyl | 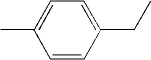 | cyclobutyl | 3-OH, 4-Ome-phenyl | 572.3 |
| 28 | 1 | 4-OMe-phenyl | 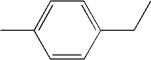 | 1-methylcyclohexyl | 4-F-phenyl | 586.3 |
| 29 | 1 | 4-OMe-phenyl | 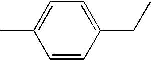 | cyclopropyl | 3-OH, 4-Ome-phenyl | 558.3 |
| 30 | 1 | 4-OMe-phenyl | 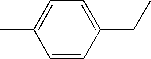 | 2-cyclopentylethyl | 4-F-phenyl | 586.3 |
| 31 | 1 | 4-OMe-phenyl | 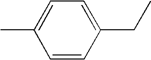 | 2-butyl | 4-F-phenyl | 546.3 |
| 32 | 1 | 4-OMe-phenyl | 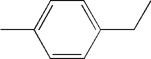 | 3-pyridyl | 4-F-phenyl | 567.3 |
| 33 | 1 | 4-OMe-phenyl | 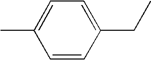 | 4-Ome-phenyl | 3-OH, 4-Ome-phenyl | 624.3 |
| 34 | 1 | 4-OMe-phenyl | 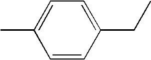 | 4-NO₂-phenyl | 3-OH, 4-Ome-phenyl | 639.3 |
| 35 | 1 | 4-OMe-phenyl | 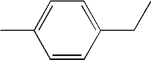 | 3-pentyl | 4-Cl-phenyl | 576.3 |
| 36 | 1 | 4-OMe-phenyl | 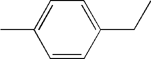 | 3-pentyl | 4-F-phenyl | 560.3 |
| 37 | 1 | 4-OMe-phenyl | 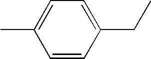 | 3-pentyl | 4-Br-phenyl | 620.2 |
| 38 | 1 | 4-OMe-phenyl | 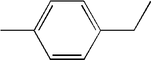 | 3-pentyl | 4-CN-phenyl | 567.3 |
| 39 | 1 | 4-OMe-phenyl | 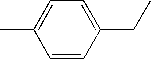 | 3-pentyl | 4-NO₂-phenyl | 587.3 |
| 40 | 1 | 4-OMe-phenyl | 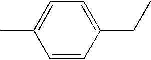 | 3-pentyl | 3-OH-phenyl | 558.3 |
| 41 | 1 | 4-OMe-phenyl | 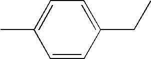 | 3-pentyl | 4-OMe-phenyl | 572.3 |
| 42 | 1 | 4-OMe-phenyl | 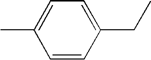 | 3-pentyl | 4-Me-phenyl | 556.4 |
| 43 | 1 | 4-OMe-phenyl | 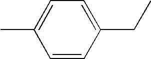 | 3-pentyl | 2,4-di-F-phenyl | 578.3 |
| 44 | 1 | 4-OMe-phenyl | 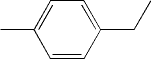 | 3-pentyl | 3-indole | 581.3 |
| 45 | 1 | 4-OMe-phenyl | 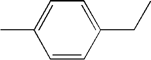 | 3-pentyl | 3-Me-phenyl | 556.4 |
| 46 | 1 | 4-OMe-phenyl | 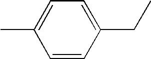 | 3-pentyl | 3-Br-phenyl | 620.2 |
| 47 | 1 | 4-OMe-phenyl | 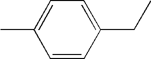 | 3-pentyl | 4-SMe-phenyl | 588.3 |
| 48 | 1 | 3-OMe-phenyl | 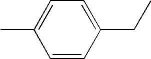 | i-propyl | 4-F-phenyl | 532.3 |
| 49 | 1 | 3-OMe-phenyl | 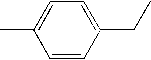 | 3-pentyl | 4-Cl-phenyl | 576.3 |
| 50 | 1 | 3-Me-phenyl | 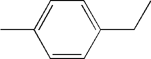 | 3-pentyl | 4-Cl-phenyl | 560.3 |
| 51 | 1 | 3-F-phenyl | 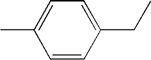 | 3-pentyl | 4-Cl-phenyl | 564.3 |
| 52 | 1 | 3-Me-phenyl | 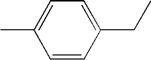 | 3-pentyl | 4-F-phenyl | 544.3 |
| 53 | 1 | 3-Me-phenyl | 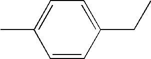 | 3-pentyl | 4-Br-phenyl | 604.3 |
| 54 | 1 | 3-F-phenyl | 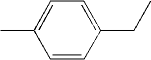 | 3-pentyl | 4-F-phenyl | 548.3 |
| 55 | 1 | 3-OMe-phenyl | 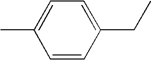 | 3-pentyl | 4-Br-phenyl | 620.2 |
| 56 | 1 | 4-Cl-phenyl | 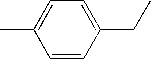 | 3-pentyl | 4-F-phenyl | 564.3 |
| 57 | 1 | 4-F-phenyl | 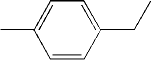 | 3-pentyl | 4-F-phenyl | 548.3 |
| 58 | 1 | 3-OMe-phenyl | 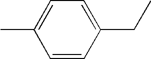 | 3-pentyl | 4-F-phenyl | 560.3 |
| 59 | 1 | 4-OMe-phenyl | 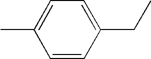 | i-propyl | 4-NO₂-phenyl | 559.3 |
| 60 | 1 | phenyl | 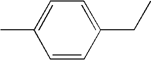 | 3-pentyl | 4-Br-phenyl | 590.2 |
| 61 | 1 | 3-Cl-phenyl | 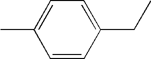 | i-propyl | 3-OH, 4-OMe-phenyl | 564.3 |
| 62 | 1 | phenyl | 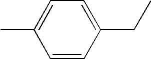 | 3-pentyl | 4-F-phenyl | 530.3 |
| 63 | 1 | 4-F-phenyl | 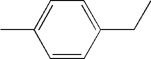 | 3-pentyl | 4-Cl-phenyl | 564.3 |
| 64 | 1 | 3-F-phenyl | 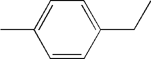 | i-propyl | 4-CN-phenyl | 527.3 |
| 65 | 1 | 4-F-phenyl | 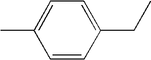 | 3-pentyl | 4-Br-phenyl | 608.2 |
| 66 | 1 | phenyl | 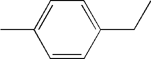 | 3-pentyl | 4-Cl-phenyl | 546.3 |
| 67 | 1 | 4-Me-phenyl | 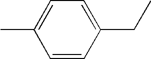 | 3-pentyl | 4-F-phenyl | 544.3 |
| 68 | 1 | 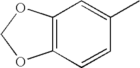 | 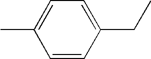 | 3-pentyl | 4-F-phenyl | 574.3 |
| 69 | 1 | 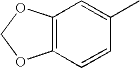 | 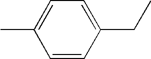 | 3-pentyl | 4-Cl-phenyl | 590.3 |
| 70 | 1 | phenyl | 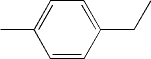 | 3-pentyl | 4-Cl-phenyl | 546.3 |
| 71 | 1 | 3,4-di-OMe-phenyl | 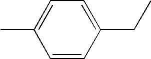 | i-propyl | 4-Cl-phenyl | 578.3 |
| 72 | 1 | 3,4-di-OMe-phenyl | 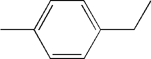 | 3-pentyl | 4-Cl-phenyl | 606.3 |
| 73 | 1 | 3,4-di-OMe-phenyl | 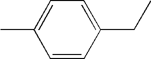 | 3-pentyl | 4-F-phenyl | 590.3 |
| 74 | 1 | 3,4-di-OMe-phenyl | 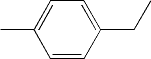 | i-propyl | 4-F-phenyl | 562.3 |
| 75 | 1 | 3,4-di-OMe-phenyl | 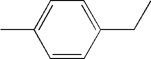 | t-butyl | 4-Cl-phenyl | 592.3 |
| 76 | 1 | 3,4-di-OMe-phenyl | 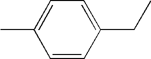 | ethyl | 4-Cl-phenyl | 564.3 |
| 77 | 1 | 3,4-di-OMe-phenyl | 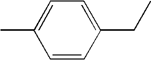 | cyclobutyl | 4-Cl-phenyl | 590.3 |
| 78 | 1 | 3,4-di-OMe-phenyl | 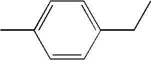 | cyclopentyl | 4-Cl-phenyl | 604.3 |
| 79 | 1 | 3,4-di-OMe-phenyl | 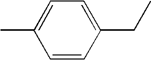 | cyclobutyl | 4-F-phenyl | 574.3 |
| 80 | 1 | 3,4-di-OMe-phenyl | 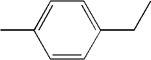 | cyclopropyl | 4-F-phenyl | 560.3 |
| 81 | 1 | 3,4-di-OMe-phenyl | 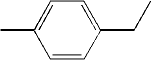 | ethyl | 4-F-phenyl | 548.3 |
| 82 | 1 | 3,4-di-OMe-phenyl | 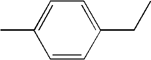 | cyclopropyl | 4-Cl-phenyl | 576.3 |
| 83 | 1 | 3,4-di-OMe-phenyl | 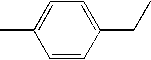 | cyclopentyl | 4-F-phenyl | 588.3 |
| 84 | 1 | 3,4-di-OMe-phenyl | 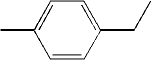 | t-butyl | 4-F-phenyl | 576.3 |
| 85 | 1 | 3,4-di-OMe-phenyl | 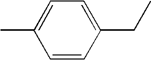 | i-propyl | 4-Br-phenyl | 622.2 |
| 86 | 1 | 3,4-di-OMe-phenyl | 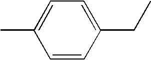 | 3-pentyl | 4-Br-phenyl | 650.3 |
| 87 | 1 | 3,4-di-OMe-phenyl | 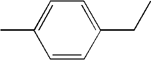 | n-propyl | 4-Cl-phenyl | 578.3 |
| 88 | 1 | 3,4-di-OMe-phenyl | 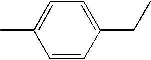 | n-propyl | 4-F-phenyl | 562.3 |
| 89 | 1 | 3,4-di-OMe-phenyl | 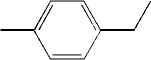 | i-propyl | 4-CN-phenyl | 569.3 |
| 90 | 1 | 3,4-di-OMe-phenyl | 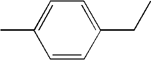 | 3-pentyl | 4-CN-phenyl | 597.3 |
| 91 | 1 | 3,4-di-OMe-phenyl | 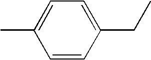 | cyclobutyl | 4-Me-phenyl | 570.3 |
| 92 | 1 | 3,4-di-OMe-phenyl | 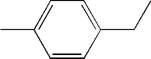 | t-butyl | 4-Br-phenyl | 636.2 |
| 93 | 1 | 3,4-di-OMe-phenyl | 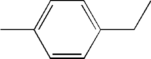 | cyclopentyl | 4-Br-phenyl | 648.2 |
| 94 | 1 | 3,4-di-OMe-phenyl | 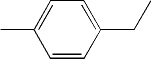 | cyclobutyl | 4-Br-phenyl | 634.2 |
| 95 | 1 | 3,4-di-OMe-phenyl | 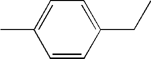 | i-propyl | 3-OH, 4-OMe-phenyl | 590.3 |
| 96 | 1 | 3,4-di-OMe-phenyl | 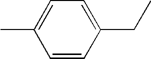 | cyclopropyl | 4-CN-phenyl | 567.3 |
| 97 | 1 | 3,4-di-OMe-phenyl | 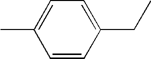 | n-propyl | 4-CN-phenyl | 569.3 |
| 98 | 1 | 3,4-di-OMe-phenyl | 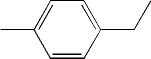 | n-propyl | 4-Br-phenyl | 622.2 |
| 99 | 1 | 3,4-di-OMe-phenyl | 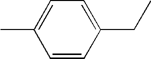 | cyclopropyl | 4-Br-phenyl | 620.2 |
| 100 | 1 | 3,4-di-OMe-phenyl | 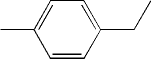 | 3-pentyl | 3-OH, 4-OMe-phenyl | 618.4 |
| 101 | 1 | 3,4-di-OMe-phenyl | 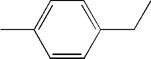 | 3-pentyl | 4-NO₂-phenyl | 617.3 |
| 102 | 1 | 3,4-di-OMe-phenyl | 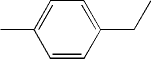 | t-butyl | 3-OH, 4-OMe-phenyl | 604.3 |
| 103 | 1 | 3,4-di-OMe-phenyl | 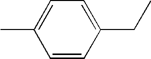 | i-propyl | 4-Me-phenyl | 558.3 |
| 104 | 1 | 3,4-di-OMe-phenyl | 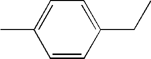 | 3-pentyl | 4-OMe-phenyl | 602.4 |
| 105 | 1 | 3,4-di-OMe-phenyl | 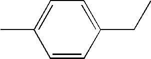 | t-butyl | 4-CN-phenyl | 583.3 |
| 106 | 1 | 3,4-di-OMe-phenyl | 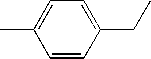 | cyclobutyl | 3-OH, 4-OMe-phenyl | 602.3 |
| 107 | 1 | 3,4-di-OMe-phenyl | 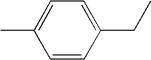 | cyclobutyl | 4-CN-phenyl | 581.3 |
| 108 | 1 | 3,4-di-OMe-phenyl | 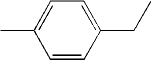 | ethyl | 4-CN-phenyl | 555.3 |
| 109 | 1 | 3,4-di-OMe-phenyl | 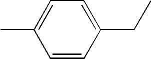 | i-propyl | 2,4-Di-F-phenyl | 580.3 |
| 110 | 1 | 3,4-di-OMe-phenyl | 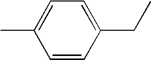 | i-propyl | 4-OMe-phenyl | 574.3 |
| 111 | 1 | 3,4-di-OMe-phenyl | 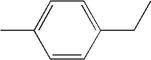 | i-propyl | 4-NO₂-phenyl | 589.3 |
| 112 | 1 | 3,4-di-OMe-phenyl | 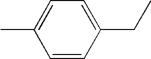 | 3-pentyl | 4-Me-phenyl | 586.4 |
| 113 | 1 | 3,4-di-OMe-phenyl | 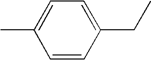 | cyclopropyl | 4-NO₂-phenyl | 587.3 |
| 114 | 1 | 3,4-di-OMe-phenyl | 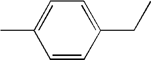 | cyclopentyl | 4-CN-phenyl | 595.3 |
| 115 | 1 | 3,4-di-OMe-phenyl | 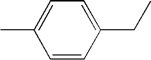 | n-propyl | 4-NO₂-phenyl | 589.3 |
| 116 | 1 | 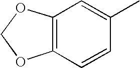 | 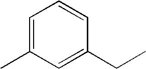 | 3-pentyl | 4-F-phenyl | 574.3 |
| 117 | 1 | 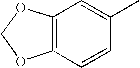 | 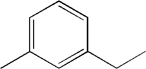 | 3-pentyl | 4-Cl-phenyl | 590.3 |
| 118 | 1 | phenyl | 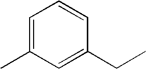 | 3-pentyl | 4-Cl-phenyl | 546.3 |
| 119 | 1 | phenyl | 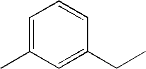 | 3-pentyl | 4-F-phenyl | 530.3 |
| 120 | 2 | 4-Cl-phenyl | —CH₂CH₂— | cyclopentylmethyl | 1-Ac-3-indole | 591.3 |
| 121 | 2 | 4-Cl-phenyl | —CH₂CH₂— | cyclopentylmethyl | 1-Me-3-indole | 563.3 |
| 122 | 2 | 4-Cl-phenyl | —CH₂CH₂— | cyclopentylmethyl | 3-OEt, 4-OH-phenyl | 570.3 |
| 123 | 2 | 4-Cl-phenyl | —CH₂CH₂— | cyclopentylmethyl | 3-indole | 549.3 |

### Utility

The utility of the compounds in accordance with the present invention as modulators of chemokine receptor activity may be demonstrated by methodology known in the art, such as the assays for CCR-2 and CCR-3 ligand binding, as disclosed by Ponath et al., J. Exp. Med., 183, 2437-2448 (1996) and Uguccioni et al., J. Clin. Invest., 100, 1137-1143 (1997). Cell lines for expressing the receptor of interest include those naturally expressing the chemokine receptor, such as EOL-3 or THP-1, those induced to express the chemokine receptor by the addition of chemical or protein agents, such as HL-60 or AML14.3D10 cells treated with, for example, butyric acid with interleukin-5 present, or a cell engineered to express a recombinant chemokine receptor, such as CHO or HEK-293. Finally, blood or tissue cells, for example human peripheral blood eosinophils, isolated using methods as described by Hansel et al., J. Immunol. Methods, 145, 105-110 (1991), can be utilized in such assays.

The utility of the compounds in accordance with the present invention as inhibitors of the migration of eosinophils or cell lines expressing the chemokine receptors may be demonstrated by methodology known in the art, such as the intracellular calcium measurement (disclosed by Bacon et al., Brit. J. Pharmacol., 95, 966-974 (1988)). In particular, the compounds of the present invention have activity in inhibition of the migration of eosinophils in the aforementioned assays. As used herein, “activity” is intended to mean a compound demonstrating an IC50 of 10 μM or lower in concentration when measured in the aforementioned assays. Such a result is indicative of the intrinsic activity of the compounds as modulators of chemokine receptor activity. An intracellular calcium measurement protocol is described below.

### Intracellular Ca²⁺ Measurement

Cells (8×10⁵/mL) were loaded with 4 μM Fluo-3 AM (Molecular Probes, Eugene. Oreg.) in calcium-free PBS containing 0.1% BSA, 1% FBS, 20 mM HEPES, 5 mM glucose and 2.5 mM probenecid) for 60 minutes at 37° C. in the dark. After two washes in buffer (PBS with 0.1% BSA, 20 mM HEPES, 5 mM glucose and 2.5 mM probenecid), cells (2×10⁶/mL) were resuspended in RPMI containing 0.1% BSA, 20 mM HEPES and 2.5 mM probenecid and plated in 96-well black, clear-bottomed plates (# 3603, Corning, Acton, Mass.), previously coated with poly-D-lysine, at 2×10⁵/well. Individual plates were inserted in a FLIPR (Molecular Devices, Sunnyvale, Calif.). Compound or vehicle (50 μL) was added robotically and incubated for 5 minutes at room temperature, then eotaxin (50 μL) was added for a final concentration of 10 nM. The eotaxin-dependent increase in fluorescence over baseline was recorded in duplicate wells.

The utility of the compounds in accordance with the present invention as inhibitors of the migration of eosinophils or cell lines expressing the chemokine receptors may be demonstrated by methodology known in the art, such as the chemotaxis assay disclosed by Bacon et al., Brit. J. Pharmacol., 95, 966-974 (1988). In particular, the compounds of the present invention have activity in inhibition of the migration of eosinophils in the aforementioned assays. As used herein, “activity” is intended to mean a compound demonstrating an IC50 of 10 μM or lower in concentration when measured in the aforementioned assays. Such a result is indicative of the intrinsic activity of the compounds as modulators of chemokine receptor activity. A human eosinophil chemotaxis assay protocol is described below.

### Human Eosinophil Chemotaxis Assay

Neuroprobe MBA96 96-well chemotaxis chambers with Neuroprobe polyvinylpyrrolidone-free polycarbonate PFD5 5-micron filters in place are warmed in a 37° C. incubator prior to assay. Freshly isolated human eosinophils, isolated according to a method such as that described by Hansel et al. (1991), are suspended in RPMI 1640 with 0.1% bovine serum albumin at 1×10⁶ cells/ml and warmed in a 37° C. incubator prior to assay. A 20 nM solution of human eotaxin in RPMI 1640 with 0.1% bovine serum albumin is warmed in a 37° C. incubator prior to assay. The eosinophil suspension and the 20 nM eotaxin solution are each mixed 1:1 with prewarmed RPMI 1640 with 0.1% bovine serum albumin with or without a dilution of a test compound that is at two fold the desired final concentration. These mixtures are warmed in a 37° C. incubator prior to assay. The filter is separated from the prewarmed Neuroprobe chemotaxis chamber and the eotaxin/compound mixture is placed into a Polyfiltronics MPC 96 well plate that has been placed in the bottom part of the Neuro Probe chemotaxis chamber. The approximate volume is 370 microliters and there should be a positive meniscus after dispensing. The filter is replaced above the 96 well plate, the rubber gasket is attached to the bottom of the upper chamber, and the chamber assembled. A 200 μl volume of the cell suspension/compound mixture is added to the appropriate wells of the upper chamber. The upper chamber is covered with a plate sealer, and the assembled unit placed in a 37° C. incubator for 45 minutes. After incubation, the plate sealer is removed and all remaining cell suspension is aspirated off. The chamber is disassembled and, while holding the filter by the sides at a 90-degree angle, unmigrated cells are washed away using a gentle stream of phosphate buffered saline dispensed from a squirt bottle and then the filter wiped with a rubber tipped squeegee. The filter is allowed to completely dry and immersed completely in Wright Giemsa stain for 30-45 seconds. The filter is rinsed with distilled water for 7 minutes, rinsed once with water briefly, and allowed to dry. Migrated cells are enumerated by microscopy.

Mammalian chemokine receptors provide a target for interfering with or promoting immune cell function in a mammal, such as a human. Compounds that inhibit or promote chemokine receptor function are particularly useful for modulating immune cell function for therapeutic purposes. Accordingly, the present invention is directed to compounds which are useful in the prevention and/or treatment of a wide variety of inflammatory, infectious, and immunoregulatory disorders and diseases, including asthma and allergic diseases, infection by pathogenic microbes (which, by definition, includes viruses), as well as autoimmune pathologies such as the rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation or infectious disease. As a result, one or more inflammatory process, such as leukocyte emigration, adhesion, chemotaxis, exocytosis (e.g., of enzymes, histamine) or inflammatory mediator release, is inhibited. For example, eosinophilic infiltration to inflammatory sites (e.g., in asthma or allergic rhinitis) can be inhibited according to the present method. In particular, the compound of the following examples has activity in blocking the migration of cells expressing the CCR-3 receptor using the appropriate chemokines in the aforementioned assays. As used herein, “activity” is intended to mean a compound demonstrating an IC₅₀ of 10 μM or lower in concentration when measured in the aforementioned assays. Such a result is also indicative of the intrinsic activity of the compounds as modulators of chemokine receptor activity.

Similarly, an instant compound which promotes one or more functions of the mammalian chemokine receptor (e.g., a human chemokine) as administered to stimulate (induce or enhance) an immune or inflammatory response, such as leukocyte emigration, adhesion, chemotaxis, exocytosis (e.g., of enzymes, histamine) or inflammatory mediator release, resulting in the beneficial stimulation of inflammatory processes. For example, eosinophils can be recruited to combat parasitic infections. In addition, treatment of the aforementioned inflammatory, allergic and autoimmune diseases can also be contemplated for an instant compound which promotes one or more functions of the mammalian chemokine receptor if one contemplates the delivery of sufficient compound to cause the loss of receptor expression on cells through the induction of chemokine receptor internalization or the delivery of compound in a manner that results in the misdirection of the migration of cells.

In addition to primates, such as humans, a variety of other mammals can be treated according to the method of the present invention. For instance, mammals, including but not limited to, cows, sheep, goats, horses, dogs, cats, guinea pigs, rats or other bovine, ovine, equine, canine, feline, rodent or murine species can be treated. However, the method can also be practiced in other species, such as avian species. The subject treated in the methods above is a mammal, male or female, in whom modulation of chemokine receptor activity is desired. “Modulation” as used herein is intended to encompass antagonism, agonism, partial antagonism and/or partial agonism.

Diseases or conditions of human or other species which can be treated with inhibitors of chemokine receptor function, include, but are not limited to: inflammatory or allergic diseases and conditions, including respiratory allergic diseases such as asthma, allergic rhinitis, hypersensitivity lung diseases, hypersensitivity pneumonitis, eosinophilic cellulitis (e.g., Well's syndrome), eosinophilic pneumonias (e.g., Loeffler's syndrome, chronic eosinophilic pneumonia), eosinophilic fasciitis (e.g., Shulman's syndrome), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), eosinophilia-myalgia syndrome due to the ingestion of contaminated tryptophan, insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such as an dermatitis, eczema, atopic dermatitis, allergic contact dermatitis, urticaria; vasculitis (e.g., necrotizing, cutaneous, and hypersensitivity vasculitis); eosinophilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis. Infectious diseases or conditions of human or other species which can be treated with inhibitors of chemokine receptor function, include, but are not limited to, HIV.

Diseases or conditions of humans or other species which can be treated with promoters of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms); (Trichuriasis, Enterobiasis, Ascariasis, Hookworm, Strongyloidiasis, Trichinosis, filariasis); trematodes (flukes) (Schistosomiasis, Clonorchiasis), cestodes (tape worms) (Echinococcosis, Taeniasis saginata, Cysticercosis); visceral worms, visceral larva migraines (e.g., Toxocara), eosinophilic gastroenteritis (e.g., Anisaki sp., Phocanema sp.), cutaneous larva migraines (Ancylostona braziliense, Ancylostoma caninum). The compounds of the present invention are accordingly useful in the prevention and treatment of a wide variety of inflammatory, infectious and immunoregulatory disorders and diseases. In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

In another aspect, the instant invention may be used to evaluate the putative specific agonists or antagonists of a G protein coupled receptor. The present invention is directed to the use of these compounds in the preparation and execution of screening assays for compounds that modulate the activity of chemokine receptors. Furthermore, the compounds of this invention are useful in establishing or determining the binding site of other compounds to chemokine receptors, e.g., by competitive inhibition or as a reference in an assay to compare its known activity to a compound with an unknown activity. When developing new assays or protocols, compounds according to the present invention could be used to test their effectiveness. Specifically, such compounds may be provided in a commercial kit, for example, for use in pharmaceutical research involving the aforementioned diseases. The compounds of the instant invention are also useful for the evaluation of putative specific modulators of the chemokine receptors. In addition, one could utilize compounds of this invention to examine the specificity of G protein coupled receptors that are not thought to be chemokine receptors, either by serving as examples of compounds which do not bind or as structural variants of compounds active on these receptors which may help define specific sites of interaction.

Combined therapy to prevent and treat inflammatory, infectious and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities. For example, in the treatment or prevention of inflammation, the present compounds may be used in conjunction with an anti-inflammatory or analgesic agent such as an opiate agonist, a lipoxygenase inhibitor, a cyclooxygenase-2 inhibitor, an interleukin inhibitor, such as an interleukin-1 inhibitor, a tumor necrosis factor inhibitor, an NMDA antagonist, an inhibitor or nitric oxide or an inhibitor of the synthesis of nitric oxide, a non-steroidal anti-inflammatory agent, a phosphodiesterase inhibitor, or a cytokine-suppressing anti-inflammatory agent, for example with a compound such as acetaminophen, aspirin, codeine, fentaynl, ibuprofen, indomethacin, ketorolac, morphine, naproxen, phenacetin, piroxicam, a steroidal analgesic, sufentanyl, sunlindac, interferon alpha and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levodesoxy-ephedrine; and antitussive such as codeine, hydrocodone, caramiphen, carbetapentane, or dextramethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) integrin antagonists such as those for selectins, ICAMs and VLA-4; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as b2-agonists (terbutaline, metaproterenol, fenoterol, isoetharine, albuteral, bitolterol, and pirbuterol), theophylline, cromolyn sodium, atropine, ipratropium bromide, leukotriene antagonists (zafirlukast, montelukast, pranlukast, iralukast, pobilukast, SKB-102,203), leukotriene biosynthesis inhibitors (zileuton, BAY-1005); (f) non-steroidal antiinflammatory agents (NSAIDs) such as propionic acid derivatives (alminoprofen, benxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors; (j) cholesterol lowering agents such as HMG-COA reductase inhibitors (lovastatin, simvastatin and pravastatin, fluvastatin, atorvsatatin, and other statins), sequestrants (cholestyramine and colestipol), nicotonic acid, fenofibric acid derivatives (gemfibrozil, clofibrat, fenofibrate and benzafibrate), and probucol; (k) anti-diabetic agents such as insulin, sulfonylureas, biguanides (metformin), a-glucosidase inhibitors (acarbose) and glitazones (troglitazone ad pioglitazone); (l) preparations of interferons (interferon alpha-2a, interferon-2B, interferon alpha-N3, interferon beta-1a, interferon beta-1b, interferon gamma-1b); (m) antiviral compounds such as efavirenz, nevirapine, indinavir, ganciclovir, lamivudine, famciclovir, and zalcitabine; (n) other compounds such as 5-aminosalicylic acid and prodrugs thereof, antimetabolites such as azathioprine and 6-mercaptopurine, and cytotoxic cancer chemotherapeutic agents. The weight ratio of the compound of the present invention to the second active ingredient may be varied and will depend upon the effective doses of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

The compounds are administered to a mammal in a therapeutically effective amount. By “therapeutically effective amount” it is meant an amount of a compound of Formula I that, when administered alone or in combination with an additional therapeutic agent to a mammal, is effective to prevent or ameliorate the thromboembolic disease condition or the progression of the disease.

### Dosage and Formulation

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder.

By way of general guidance, the daily oral dosage of each active ingredient, when used for the indicated effects, will range between about 0.001 to 1000 mg/kg of body weight, preferably between about 0.01 to 100 mg/kg of body weight per day, and most preferably between about 1.0 to 20 mg/kg/day. Intravenously, the most preferred doses will range from about 1 to about 10 mg/kg/minute during a constant rate infusion. Compounds of this invention may be administered in a single daily dose, or the total daily dosage may be administered in divided doses of two, three, or four times daily.

Compounds of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

Representative useful pharmaceutical dosage-forms for administration of the compounds of this invention can be illustrated as follows:

### Capsules

A large number of unit capsules can be prepared by filling standard two-piece hard gelatin capsules each with 100 milligrams of powdered active ingredient, 150 milligrams of lactose, 50 milligrams of cellulose, and 6 milligrams magnesium stearate.

### Soft Gelatin Capsules

A mixture of active ingredient in a digestable oil such as soybean oil, cottonseed oil or olive oil may be prepared and injected by means of a positive displacement pump into gelatin to form soft gelatin capsules containing 100 milligrams of the active ingredient. The capsules should be washed and dried.

### Tablets

Tablets may be prepared by conventional procedures so that the dosage unit is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

### Injectable

A parenteral composition suitable for administration by injection may be prepared by stirring 1.5% by weight of active ingredient in 10% by volume propylene glycol and water. The solution should be made isotonic with sodium chloride and sterilized.

### Suspension

An aqueous suspension can be prepared for oral administration so that each 5 mL contain 100 mg of finely divided active ingredient, 200 mg of sodium carboxymethyl cellulose, 5 mg of sodium benzoate, 1.0 g of sorbitol solution, U.S.P., and 0.025 mL of vanillin.

Where the compounds of this invention are combined with other anticoagulant agents, for example, a daily dosage may be about 0.1 to 100 milligrams of the compound of Formula I and about 1 to 7.5 milligrams of the second anticoagulant, per kilogram of patient body weight. For a tablet dosage form, the compounds of this invention generally may be present in an amount of about 5 to 10 milligrams per dosage unit, and the second anti-coagulant in an amount of about 1 to 5 milligrams per dosage unit.

Where two or more of the foregoing second therapeutic agents are administered with the compound of Formula I, generally the amount of each component in a typical daily dosage and typical dosage form may be reduced relative to the usual dosage of the agent when administered alone, in view of the additive or synergistic effect of the therapeutic agents when administered in combination.

Particularly when provided as a single dosage unit, the potential exists for a chemical interaction between the combined active ingredients. For this reason, when the compound of Formula I and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

While the invention has been described in detail and with reference to specific embodiments thereof, it will be apparent to one skilled in the art that various changes and modifications can be made therein without departing from the spirit and scope thereof.

What is claimed:

1. A compound of formula (I): 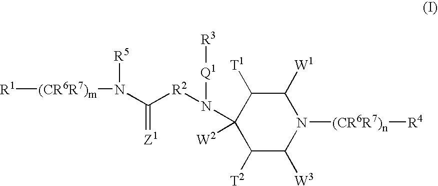
or a stereoisomer or prodrug or pharmaceutically acceptable salt from thereof, wherein:
Q¹ is independently selected from C═O, C(O)₂, CON(R⁸)R⁸, S(O), S(O)₂ and S(O)₂N(R¹⁸)R⁸;
Z¹ is independently selected from O, S, N(R⁸), C(CN)₂, CH(NO₂), and CH(CN);
R¹ is independently selected from hydrogen, C₁-C₆ alkyl, (CH₂)_{q}C₃-C₆ cycloalkyl, C₃-C₆ cycloalkyl(CH₂)ᵣ, aryl and heterocyclyl, wherein the C₁-C₆ alkyl, (CH₂)_{q}C₃-C₆ cycloalkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁸)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;
R² is independently selected from C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ, wherein the C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;
R³ is independently selected from hydrogen, C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ wherein the C₁-C₆ alkyl, (CH₂)_{q}C₃-C₆ cycloalkyl, (CH₂)_{q}aryl, (CH₂)_{q}heterocyclyl, C₃-C₆ cycloalkyl(CH₂)ᵣ, aryl(CH₂)ᵣ and heterocyclyl(CH₂)ᵣ may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;
R⁴ is independently selected from C₁-C₆ alkyl, (CH₂)_{q}C₃-C₆ cycloalkyl, C₃-C₆ cycloalkyl(CH₂)ᵣ, aryl and heterocyclyl, wherein the C₁-C₆ alkyl, (CH₂)_{q}C₃-C₆ cycloalkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;
R⁵ is selected from hydrogen, C₁₋₈ alkyl, C₃₋₈ alkenyl, C₃₋₈ alkynyl, (CH₂)ᵣC₃₋₆ cycloalkyl, (CH₂)_{q}C(O)R¹⁶, (CH₂)_{q}C(O)NR¹⁵R¹⁵, (CH₂)_{q}C(O)OR¹⁶, (CH₂)ᵣaryl optionally substituted with 0-3 R¹⁷, and (CH₂)ᵣheterocyclyl optionally substituted with 0-3 R¹⁷;
R⁶ and R⁷, at each occurrence, are independently selected from hydrogen, C₁₋₆ alkyl, (CH₂)ᵣC₃₋₆ cycloalkyl, and (CH₂)ᵣphenyl, or optionally, R⁶ and R⁷ may be taken together with the carbon to which both are attached to form a C₃₋₆ cycloalkyl ring;
R⁸, at each occurrence, is independently selected from hydrogen, C₁-C₆ alkyl, C₂₋₈ alkenyl, C₂₋₈ alkynyl, (CH₂)ᵣC₃-C₆ cycloalkyl, (CH₂)ᵣC(O)R¹⁹, (CH₂)ᵣC(O)N(R^{18a})R^{18b}, (CH₂)ᵣC(O)OR¹⁹, (CH₂)ᵣS(O)₂R¹⁹, (CH₂)ᵣS(O)₂N(R^{18a})R^{18b}, OR¹⁹, CN, NO₂, and (CH₂)ᵣphenyl substituted with 0-3 R¹⁸;
R⁹, at each occurrence, is independently selected from hydrogen, C₁₋₆ alkyl, C₂₋₈ alkenyl, C₂₋₈ alkynyl, (CH₂)ᵣaryl substituted with 0-5 R¹³, and a (CH₂)ᵣheterocyclyl containing 1-4 heteroatoms selected from N, O, and S, substituted with 0-2 R¹³;
R¹⁰, at each occurrence, is independently selected from C₁₋₆ alkyl, C₂₋₈ alkenyl, C₂₋₈ alkynyl, (CH₂)ᵣaryl substituted with 0-3 R¹³, and a (CH₂)ᵣheterocyclyl containing 1-4 heteroatoms selected from N, O, and S, substituted with 0-2 R¹³;
R¹², at each occurrence, is independently selected from C₂₋₈ alkenyl, C₂₋₈ alkynyl, C₁₋₆ alkyl substituted with 0-3 R¹³, (CH₂)ᵣaryl substituted with 0-3 R¹³, and a (CH₂)ᵣheterocyclyl containing 1-4 heteroatoms selected from N, O, and S, substituted with 0-3 R¹³;
R¹³, at each occurrence, is independently selected from C₁₋₆ alkyl, C₂₋₈ alkenyl, C₂₋₈ alkynyl, (CH₂)ᵣC₃₋₆ cycloalkyl, Cl, F, Br, I, CN, NO₂, (CF₂)ᵣCF₃, (CH₂)ᵣOC₁₋₅ alkyl, OH, SH, (CH₂)ᵣSC₁₋₁₅ alkyl, and (CH₂)ᵣNR¹⁴R¹⁴;
R¹⁴, at each occurrence, is independently selected from H, C₁₋₅ alkyl, and C₃₋₆ cycloalkyl, and phenyl;
R¹⁵, at each occurrence, is independently selected from H, C₁₋₆ alkyl, (CH₂)ᵣC₃₋₆ cycloalkyl, and (CH₂)ᵣphenyl;
R¹⁶, at each occurrence, is independently selected from C₁₋₆ alkyl, C₂₋₈ alkenyl, (CH₂)ᵣC₃₋₆ cycloalkyl, C₂₋₈ alkynyl, and (CH₂)ᵣphenyl;
R¹⁷, at each occurrence, is independently selected from C₁₋₆ alkyl, C₂₋₈ alkenyl, C₂₋₈ alkynyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣNR¹⁵R¹⁵, and (CH₂)ᵣphenyl;
R¹⁸, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₁₅ alkyl, (CH₂)ᵣS(O)C₁₋₅ alkyl, (CH₂)ᵣS(O)₂C₁₋₁₅ alkyl, (CH₂)ᵣS(O)₂N(R^{18a})R^{18b}, (CH₂)ᵣN(R^{18c})C(O)C₁₋₅ alkyl (CH₂)ᵣN(R^{18c})S(O)₂C₁₋₅ alkyl, (CH₂)ᵣC(O)N(R^{18a})R^{18b}, (CH₂)ᵣC(O)OC₁₋₅ alkyl, (CH₂)ᵣC(O)C₁₋₅ alkyl, and (CH₂)ᵣN(R^{18a})R^{18b};
R^{18a}, R^{18b}, and R^{18c}, at each occurrence, are independently selected from H, C₁₋₆ alkyl, (CH₂)ᵣC₃₋₆ cycloalkyl, and (CH₂)ᵣphenyl;
R¹⁹, at each occurrence, is independently selected from hydrogen, C₁₋₆ alkyl, (CH₂)ᵣC₃₋₆ cycloalkyl, and (CH₂)ᵣphenyl;
T¹ and T² are independently selected from hydrogen, C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;
W¹, W² and W³ are independently selected from hydrogen, C₁₋₈ alkyl, (CH₂)ᵣC₃₋₆ cycloalkyl, (CHR⁸)_{q}NR⁹R⁹, (CHR⁸)_{q}OH, (CHR⁸)_{q}O(CHR⁸)ᵣR¹², (CHR⁸)_{q}SH, (CHR⁸)ᵣC(O)H, (CHR⁸)_{q}S(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)_{q}NR¹⁴C(O)(CHR⁸)ᵣR¹, (CHR⁸)ᵣC(O)O(CHR⁸)ᵣR¹², (CHR⁸)_{q}OC(O)(CHR⁸)ᵣR¹⁰, (CHR⁸)ᵣC(═NR¹⁴)NR⁹R⁹, (CHR⁸)_{q}NHC(═NR¹⁴)NR¹⁴R¹⁴, (CHR⁸)_{q}S(O)(CHR⁸)ᵣR¹⁰, (CHR⁸)_{q}S(O)₂(CHR⁸)ᵣR¹⁰, (CHR⁸)_{q}S(O)₂NR⁹R⁹, (CHR⁸)_{q}NR¹⁴S(O)₂(CHR⁸)ᵣR¹⁰, C₁₋₆ haloalkyl, C₂₋₈ alkenyl, C₂₋₈ alkynyl, and (CH₂)ᵣphenyl substituted with 0-3 R¹³;
m is 0-3;
n is 0-3;
q is 1-5; and
r is 0-5.

2. The compound of claim 1, wherein:
Q¹ is independently selected from C═O, C(O)₂, and CON(R⁸)R⁸;
Z¹ is independently selected from O and S;
R¹ is independently selected from aryl and heterocyclyl, wherein the aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R² is independently selected from C₁-C₆ alkyl, C₃-C₆ cycloalkyl(CH₂)ᵣ and aryl(CH₂)ᵣ, wherein the C₁-C₆ alkyl, C₃-C₆ cycloalkyl(CH₂)ᵣ and aryl(CH₂)ᵣ may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R³ is independently selected from hydrogen, C₁-C₆ alkyl, C₃-C₆ cycloalkyl(CH₂)ᵣ, aryl and heterocyclyl, wherein the C₁-C₆ alkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R⁴ is independently selected from C₃-C₆ cycloalkyl(CH₂)ᵣ, aryl and heterocyclyl, wherein the C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)N—R⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R⁵ is selected from hydrogen and C₁₋₈ alkyl;
R⁶ and R⁷, at each occurrence, are independently selected from hydrogen and C₁₋₆ alkyl;
R⁸, at each occurrence, is independently selected from hydrogen, C₁-C₆ alkyl, (CH₂)ᵣC₃-C₆ cycloalkyl, (CH₂)ᵣC(O)R¹⁹, (CH₂)ᵣC(O)N(R^{18a})R^{18b}, (CH₂)ᵣC(O)OR¹⁹, (CH₂)ᵣS(O)₂R¹⁹, (CH₂)ᵣS(O)₂N(R^{18a})R^{18b}, OR¹⁹, CN, NO₂, and (CH₂)ᵣphenyl substituted with 0-3 R¹⁸;
R⁹, at each occurrence, is independently selected from hydrogen, C₁₋₆ alkyl, (CH₂)ᵣaryl substituted with 0-5 R¹³, and a (CH₂)ᵣheterocyclyl containing 1-4 heteroatoms selected from N, O, and S, substituted with 0-2 R¹³;
R¹⁰, at each occurrence, is independently selected from C₁₋₆ alkyl, (CH₂)ᵣaryl substituted with 0-3 R¹³, and a (CH₂)ᵣheterocyclyl containing 1-4 heteroatoms selected from N, O, and S, substituted with 0-2 R¹³;
R¹², at each occurrence, is independently selected from C₁₋₆ alkyl substituted with 0-3 R¹³, (CH₂)ᵣaryl substituted with 0-3 R¹³, and a (CH₂)ᵣheterocyclyl containing 1-4 heteroatoms selected from N, O, and S, substituted with 0-3 R¹³;
R¹³, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, OH, SH, (CH₂)ᵣSC₁₋₅ alkyl, and (CH₂)ᵣNR¹⁴R¹⁴;
R¹⁴, at each occurrence, is independently selected from H, C₁₋₅ alkyl, C₃₋₆ cycloalkyl and phenyl;
R¹⁵, at each occurrence, is independently selected from H, C₁₋₆ alkyl, (CH₂)ᵣC₃₋₆ cycloalkyl, and (CH₂)ᵣphenyl;
R¹⁶, at each occurrence, is independently selected from C₁₋₆ alkyl, (CH₂)ᵣC₃₋₆ cycloalkyl and (CH₂)ᵣphenyl;
R¹⁷, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣNR¹⁵R¹⁵ and (CH₂)ᵣphenyl;
R¹⁸, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣS(O)C₁₋₅ alkyl, (CH₂)ᵣS(O)₂C₁₋₅ alkyl, (CH₂)ᵣS(O)₂N(R^{18a})R^{18b}, (CH₂)ᵣN(R^{18c})C(O)C₁₋₅ alkyl (CH₂)ᵣN(R^{18c})S(O)₂C₁₋₅ alkyl, (CH₂)ᵣC(O)N(R^{18a})R^{18b}, (CH₂)ᵣC(O)OC₁₋₅ alkyl, (CH₂)ᵣC(O)C₁₋₅ alkyl, and (CH₂)ᵣN(R^{18a})R^{18b};
R^{18a}, R^{18b}, and R^{18c}, at each occurrence, are independently selected from H, C₁₋₆ alkyl, (CH₂)ᵣC₃₋₆ cycloalkyl and (CH₂)ᵣphenyl;
R¹⁹, at each occurrence, is independently selected from hydrogen, C₁₋₆ alkyl (CH₂)ᵣC₃₋₆ cycloalkyl, and (CH₂)ᵣphenyl;
T¹ and T² are independently selected from hydrogen, C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, and (CH₂)ᵣphenyl substituted with 0-3 R¹³;
W¹, W² and W³ are independently selected from hydrogen, C₁₋₈ alkyl, (CH₂)ᵣC₃₋₆ cycloalkyl, (CHR⁸)_{q}NR⁹R⁹, (CHR⁸)_{q}OH, (CHR⁸)_{q}O(CHR⁸)ᵣR¹², (CHR⁸)_{q}SH, (CHR⁸)ᵣC(O)H, (CHR⁸)_{q}S(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)_{q}NR¹⁴C(O)(CHR⁸)ᵣR¹⁰, (CHR⁸)ᵣC(O)O(CHR⁸)ᵣR¹², (CHR⁸)_{q}OC(O)(CHR⁸)ᵣR¹⁰, (CHR⁸)ᵣC(═NR¹⁴)NR⁹R⁹, (CHR⁸)_{q}NHC(═NR¹⁴)NR¹⁴R¹⁴, (CHR⁸)_{q}S(O)(CHR⁸)ᵣR¹⁰, (CHR⁸)_{q}S(O)₂(CHR⁸)ᵣR¹⁰, (CHR⁸)_{q}S(O)₂NR⁹R⁹, (CHR⁸)_{q}NR¹⁴S(O)₂(CHR⁸)ᵣR¹⁰, C₁₋₆ haloalkyl, and (CH₂)ᵣphenyl substituted with 0-3 R¹³;
m is 0-2;
n is 0-2;
q is 1-2; and
r is 0-2.

3. The compound of claim 1, wherein:
Q¹ is C═O;
Z¹ is O;
R¹ is aryl, wherein the aryl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R² is independently selected from C₁-C₆ alkyl and aryl, wherein the C₁-C₆ alkyl and aryl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R³ is independently selected from C₁-C₆ alkyl, C₃-C₆ cycloalkyl(CH₂)ᵣ, aryl and heterocyclyl, wherein the C₁-C₆ alkyl, C₃-C₆ cycloalkyl(CH₂)ᵣ, aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3R¹³;
R⁴ is independently selected from aryl and heterocyclyl, wherein the aryl and heterocyclyl may be optionally substituted with one or more substituents selected from C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl and (CH₂)ᵣphenyl substituted with 0-3 R¹³;
R⁵ is hydrogen;
R⁶ and R⁷, at each occurrence, are hydrogen;
R⁸, at each occurrence, is independently selected from hydrogen, C₁-C₆ alkyl, (CH₂)ᵣC₃-C₆ cycloalkyl, (CH₂)ᵣC(O)R¹⁹, (CH₂)ᵣC(O)N(R^{18a})R^{18b}, (CH₂)ᵣC(O)OR¹⁹, (CH₂)ᵣS(O)₂R¹⁹, (CH₂)ᵣS(O)₂N(R^{18a})R^{18b}, OR¹⁹, CN, NO₂, and (CH₂)ᵣphenyl substituted with 0-3 R¹⁸;
R⁹, at each occurrence, is independently selected from hydrogen, C₁₋₆ alkyl, (CH₂)ᵣaryl substituted with 0-5 R¹³, and a (CH₂)ᵣheterocyclyl containing 1-4 heteroatoms selected from N, O, and S, substituted with 0-2 R¹³;
R¹⁰, at each occurrence, is independently selected from C₁₋₆ alkyl, (CH₂)ᵣaryl substituted with 0-3 R¹³, and a (CH₂)ᵣheterocyclyl containing 1-4 heteroatoms selected from N, O, and S, substituted with 0-2 R¹³;
R¹², at each occurrence, is independently selected from C₁₋₆ alkyl substituted with 0-3 R¹³, (CH₂)ᵣaryl substituted with 0-3 R¹³, and a (CH₂)ᵣheterocyclyl containing 1-4 heteroatoms selected from N, O, and S, substituted with 0-3 R¹³;
R¹³, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, OH, SH, (CH₂)ᵣSC₁₋₅ alkyl, and (CH₂)ᵣNR¹⁴R¹⁴;
R¹⁴, at each occurrence, is independently selected from H, C₁₋₅ alkyl, C₃₋₆ cycloalkyl and phenyl;
R¹⁵, at each occurrence, is independently selected from H, C₁₋₆ alkyl, (CH₂)ᵣC₃₋₆ cycloalkyl, and (CH₂)ᵣphenyl;
R¹⁶, at each occurrence, is independently selected from C₁₋₆ alkyl, (CH₂)ᵣC₃₋₆ cycloalkyl and (CH₂)ᵣphenyl;
R¹⁷, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣNR¹⁵R¹⁵ and (CH₂)ᵣphenyl;
R¹⁸, at each occurrence, is independently selected from C₁₋₆ alkyl, (CH₂)ᵣC₃₋₆ cycloalkyl, Cl, F, Br, I, CN, NO₂, (CF₂)ᵣCF₃, (CH₂)ᵣOC₁₋₅ alkyl, (CH₂)ᵣOH, (CH₂)ᵣSC₁₋₅ alkyl, (CH₂)ᵣS(O)C₁₋₅ alkyl, (CH₂)ᵣS(O)₂C₁₋₁₅ alkyl, (CH₂)ᵣS(O)₂N(R^{18a})R^{18b}, (CH₂)ᵣN(R^{18c})C(O)C₁₋₅ alkyl (CH₂)ᵣN(R^{18c})S(O)₂C₁₋₅ alkyl, (CH₂)ᵣC(O)N(R^{18a})R^{18b}, (CH₂)ᵣC(O)OC₁₋₅ alkyl, (CH₂)ᵣC(O)C₁₋₅ alkyl, and (CH₂)ᵣN(R^{18a})R^{18b};
R^{18a}, R^{18b}, and R^{18c}, at each occurrence, are independently selected from H, C₁₋₆ alkyl, (CH₂)ᵣC₃₋₆ cycloalkyl and (CH₂)ᵣphenyl;
R¹⁹, at each occurrence, is independently selected from hydrogen, C₁₋₆ alkyl (CH₂)ᵣC₃₋₆ cycloalkyl, and (CH₂)ᵣphenyl;
T¹ and T² are independently selected from hydrogen, C₁₋₈ alkyl, (CH₂)ᵣC₃₋₆ cycloalkyl, Cl, Br, I, F, NO₂, CN, (CHR⁸)ᵣNR⁹R⁹, (CHR⁸)ᵣOH, (CHR⁸)ᵣO(CHR⁸)ᵣR¹², (CHR⁸)ᵣSH, (CHR⁸)ᵣC(O)H, (CHR⁸)ᵣS(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)ᵣNR¹⁴C(O)(CHR⁸)ᵣR¹⁰, (CHR⁸)ᵣC(O)O(CHR⁸)ᵣR¹², (CHR⁸)ᵣOC(O)(CHR⁸)ᵣR¹⁰, (CHR⁸)ᵣC(═NR¹⁴)NR⁹R⁹, (CHR⁸)ᵣNHC(═NR¹⁴)NR¹⁴R¹⁴, (CHR⁸)ᵣS(O)(CHR⁸)ᵣR¹⁰, (CHR⁸)ᵣS(O)₂(CHR⁸)ᵣR¹⁰, (CHR⁸)ᵣS(O)₂NR⁹R⁹, (CHR⁸)ᵣNR¹⁴S(O)₂(CHR⁸)ᵣR¹⁰, C₁₋₆ haloalkyl, and (CH₂)ᵣphenyl substituted with 0-3 R¹³;
W¹, W² and W³ are independently selected from hydrogen, C₁₋₈ alkyl, (CH₂)ᵣC₃₋₆ cycloalkyl, (CHR⁸)_{q}NR⁹R⁹, (CHR⁸)_{q}OH, (CHR⁸)_{q}O(CHR⁸)ᵣR¹², (CHR⁸)_{q}SH, (CHR⁸)ᵣC(O)H, (CHR⁸)_{q}S(CHR⁸)ᵣR¹², (CHR⁸)ᵣC(O)OH, (CHR⁸)ᵣC(O)(CHR⁶)ᵣR¹⁰, (CHR⁸)ᵣC(O)NR⁹R⁹, (CHR⁸)_{q}NR¹⁴C(O)(CHR⁸)ᵣR¹⁰, (CHR⁸)ᵣC(O)O(CHR⁸)ᵣR¹², (CHR⁸)_{q}OC(O)(CHR⁸)ᵣR¹⁰, (CHR⁸)ᵣC(═NR¹⁴)NR⁹R⁹, (CHR⁸)_{q}NHC(═NR¹⁴)NR¹⁴R¹⁴, (CHR⁸)_{q}S(O)(CHR⁸)ᵣR¹⁰, (CHR⁸)_{q}S(O)₂(CHR⁸)ᵣR¹⁰, (CHR⁸)_{q}S(O)₂NR⁹R⁹, (CHR⁸)_{q}NR¹⁴S(O)₂(CHR⁸)ᵣR¹⁰, C₁₋₆ haloalkyl, and (CH₂)ᵣphenyl substituted with 0-3 R¹³;
m is 1-2;
n is 1-2;
q is 1-2; and
r is 0-2.

4. The compound of claim 3, wherein:
R³ is independently selected from C₁-C₆ alkyl and C₃-C₆ cycloalkyl;
R⁴ is aryl, wherein the aryl may be optionally substituted with one or more substituents selected from C₁₋₈ alkyl, C₃₋₆ cycloalkyl, Cl, Br, I, F, NO₂, CN, C₁₋₆ haloalkyl and phenyl substituted with 0-3 R¹³;
R¹³, at each occurrence, is independently selected from C₁₋₆ alkyl, C₃₋₆ cycloalkyl, Cl, F, Br, I, CN, NO₂, OH, and SH;
T¹ and T² are independently selected from hydrogen, C₁₋₈ alkyl, C₃₋₆ cycloalkyl, Cl, Br, I, F, NO₂, and CN;
W¹, W² and W³ are independently selected from hydrogen, C₁₋₈ alkyl and C₃₋₆ cycloalkyl;
m is 1; and
n is 1.

5. A pharmaceutical composition, comprising a pharmaceutically acceptable carrier and a therapeutically effective amount of a compound according to claim 1, or a pharmaceutically acceptable salt thereof.

6. A method for modulation of chemokine receptor activity comprising administering to a patient in need thereof a therapeutically effective amount of a compound according to claim 1.

7. A method for treating asthma, comprising administering to a patient in need thereof a therapeutically effective amount of a compound according to claim 1.

8. The method of claim 7 wherein modulation of chemokine receptor activity comprises contacting a CCR3 receptor with an effective inhibitory amount of the compound.

9. A method for treating inflammatory disorders comprising administering to a patient in need thereof a therapeutically effective amount of a compound according to claim 1, or a pharmaceutically acceptable salt thereof.

10. A method according to claim 9, wherein the disorder is selected from asthma, allergic rhinitis, atopic dermatitis, inflammatory bowel diseases, idiopathic pulmonary fibrosis, bullous pemphigoid, helminthic parasitic infections, allergic colitis, eczema, conjunctivitis, transplantation, familial eosinophilia, eosinophilic cellulitis, eosinophilic pneumonias, eosinophilic fasciitis, eosinophilic gastroenteritis, drug induced eosinophilia, HIV infection, cystic fibrosis, Churg-Strauss syndrome, lymphoma, Hodgkin's disease, and colonic carcinoma.

11. The method according to claim 10, wherein the disorder is selected from asthma, allergic rhinitis, atopic dermatitis, and inflammatory bowel diseases.

12. A pharmaceutical composition comprised of a compound of claim 1 and one or more active ingredients.

13. A method for treating inflammatory disorders comprising administering to a patient in need thereof a therapeutically effective amount of a composition according to claim 12.

14. A method according to claim 13, wherein the disorder is selected from asthma, allergic rhinitis, atopic dermatitis, inflammatory bowel diseases, idiopathic pulmonary fibrosis, bullous pemphigoid, helminthic parasitic infections, allergic colitis, eczema, conjunctivitis, transplantation, familial eosinophilia, eosinophilic cellulitis, eosinophilic pneumonias, eosinophilic fasciitis, eosinophilic gastroenteritis, drug induced eosinophilia, HIV infection, cystic fibrosis, Churg-Strauss syndrome, lymphoma, Hodgkin's disease, and colonic carcinoma.

15. The method according to claim 14, wherein the disorder is selected from asthma, allergic rhinitis, atopic dermatitis, and inflammatory bowel diseases.
